(19) United States
(12) Patent Application Publication
(10) Pub. No.: US 2002/0094528 A1
(43) Pub. Date: Jul. 18, 2002

(54) Method and apparatus using a surface-selective nonlinear optical technique for detection of probe-target interations

(76) Inventor: Joshua S. Salafsky, New York, NY (US)

Correspondence Address: Henry D. Coleman, Coleman Sudol Sapone, P.C., 714 Colorado Avenue, Bridgeport, CT, 06605-1601, US

(21) Appl. No.: 09/907,035

(22) Filed: Jul. 17, 2001

Related U.S. Application Data
(60) Provisional application No. 60/253,862, filed on Nov. 29, 2000.
(60) Provisional application No. 60/260,249, filed on Jan. 8, 2001.
(60) Provisional application No. 60/265,775, filed on Feb. 1, 2001.
(60) Provisional application No. 60/278,941, filed on Jan. 27, 2001.

(51) Int. Cl. C12Q 1/68; C12M 1/34

(52) U.S. Cl. 435/6; 435/287.2

(57) ABSTRACT

A surface-selective nonlinear optical technique, such as second harmonic or sum frequency generation, is used to detect reactions between surface-attached probes and labeled targets or used to perform imaging of a surface. The surface-selective optical technique allows detection of only those target components near the interface while ignoring those present in the sample bulk. In addition, the direction of the nonlinear light is scattered from the surface in a well-defined direction and because of this, its incidence at a detector some distance from the surface may be easily mapped to a specific and known location on the surface.

### RELATED APPLICATIONS

This application claims priority from provisional applications 60/253,862, entitled “Method and Apparatus Using a a Surface-Selective Nonlinear Optical Technique for Detection of Probe-Target Interactions”, filed Nov. 29, 2000; 60/260,249, entitled “Apparatus and Method for the Detection of Biological Reactions Using a Surface-Selective Nonlinear Optical Technique”, filed Jan. 8, 2001; 60/265,775, entitled “Apparatus and Method for the Detection of Biological Reactions Using a Surface-Selective Nonlinear Optical Technique”, filed Feb. 1, 2001; and 60/278,941, entitled “Apparatus and Method for the Detection of Biological Reactions Using a Surface-Selective Nonlinear Optical Technique”, filed Mar. 27, 2001 (accorded a filing date of Jan. 27, 2001), the sum and substance of such applications being incorporated by reference hereof.

### FIELD OF THE INVENTION

The present invention relates to a method and apparatus for detecting interactions between biological components using a surface-selective nonlinear optical technique. In particular, the present invention relates to detection of the binding between biological probes and nonlinear-active labeled targets.

### BACKGROUND OF THE INVENTION

Detecting and quantifying interactions such as binding between biomolecules is of central interest in modern molecular biology and medicine. Genomics and proteomics research is increasingly directed toward this problem, which demands high-throughput analysis of a variety of biological interactions. Many schemes for doing this rely on immobilization of molecules, often oligonucleotides or proteins, to solid surfaces. In particular, a microarray format of samples can be used to obtain information in a highly parallel process. For example, Fodor et al. (1991, relevant portions of which are incorporated by reference herein) disclose high density arrays formed by light-directed synthesis—in this case, the surface-attached probes are oligonucleotides and are tested for binding (hybridization) against targets. The targets, freely diffusing in solution, are fluorescently-labeled oligonucleotides and at places where the nucleotide sequence of the probe matches the sequence of the target, binding occurs. When non-bound targets are removed by washing, the sequence of the remaining targets can be determined by scanning the surface for fluorescence since the probe sequence is known, by design, at each location on the surface, and targets and probes must have matching, complementary sequences to hybridize. A number of variations on this method have been introduced including: studying SNPs (single nucleotide polymorphisms), where the binding strength, and hence the fluorescence intensity, between sequences differing by one base-pair; detection of protein-protein interactions, where one protein (the probe) is immobilized to the surface and tested for binding against a variety of targets; protein-drug interactions where protein-protein interactions are modulated by the presence of a drug; and others.

In all these cases, the read-out step involves fluorescence-based detection. However, detection with fluorescence has several drawbacks: the samples are generally dry (to remove background fluorescence; i.e., non-bound targets in the bulk) and therefore no equilibrium (free energy, dissociation constant, etc.) measurement is typically possible due to fluorescence background from the bulk. The non-bound targets must first be removed from the sample via a wash step and this obviates equilibrium or kinetics measurements and, furthermore, can be time-consuming when many scans must be made on a given sample or many samples must be examined. The excitation source for fluorescence may also contribute a background signal since it can be scattered by the substrate into the detection optics, and may be difficult to completely filter from the fluorescence. Furthermore, there may be background autofluorescence or bottlenecks in the “read-out” or detection step because the scan can require pixel-by-pixel acquisition with, for example, confocal-based detection schemes. Auto-focusing routines at each step in the scan can also lead to significant slow-down in image acquisition.

Another method for quantifying biomolecular binding interactions—with an ability to measure both equilibrium and kinetics properties of the interaction is surface plasmon resonance (SPR). SPR requires a conductive or semiconductive layer (typically gold) between the substrate (typically glass) and the liquid solution it is immersed in. Incident light is coupled into the conductive layer by means of a prism or grating and, at a specific wavelength or angle of incidence, a resonance occurs, resulting in a sharp minimum or decrease in reflectivity. Generally, a bio-compatible layer or layers are built on top of the conductive layer. In one example, proteins are immobilized to the biocompatible layer (often dextran-based) and target proteins are brought into contact with the layer. The resonance wavelength or angle depends on the refractive index of the solution near the substrate and this, in turn, depends on the amount and mass of adsorbed biomolecules within an evanescent wavelength from the conductive layer. When target protein binds to the immobilized protein, a change in the resonance wavelength or angle occurs. However, the SPR technique is not convenient for detecting samples in an array format because of the difficulty in coupling the excitation into each array element separately. Furthermore, the detection sensitivity may be low, the technique cannot distinguish between specific and non-specific binding, and SPR typically requires an extra, biologically compatible layer to prevent destructive interactions which can occur if the biomolecules make contact with the conductive layer. This biocompatible layer may not always be stable or prevent destructive interactions with the gold surface and the immobilized proteins must often be truncated in order to render them suitable for coupling to the bio-compatible layer, thus risking the possibility that their properties may change. A particularly acute problem occurs with membrane proteins. Membrane proteins are best studied in a native-like environment such as a laterally fluid phospholipid membrane which can be prepared on glass surfaces. However, it is not possible to prepare these membranes on gold surfaces due to destructive interactions between the gold and the lipids.

Surface-selective nonlinear optical (SSNLO) techniques such as second harmonic generation (SHG) allow one to detect interfacial molecules or particles (the interface must be non-centrosymmetric) in the presence of the bulk species. An intense laser beam (the fundamental) is directed on to the interface of some sample; if the interface is non-centrosymmetric, the sample is capable of generating nonlinear light, i.e. the harmonics of the fundamental. The fundamental or the second harmonic beams can easily be separated from each other, unlike the typical case in fluorescence techniques with excitation and emission light, which are separated more narrowly by the Stokes shift. Individual molecules or particles can be detected if they 1) are nonlinearly active (possess a hyperpolarizability) and 2) are near to the surface and through its influence (via chemical or electric forces) become non-randomly oriented. This net orientation and the intrinsic SHG-activity of the species are responsible for an SHG-allowed effect at the interface. For example, the adsorption processes of dye molecules to planar solid surfaces (glass and silica), liposomes and solid beads (silica and polystyrene) at air-water interface have been measured. The technique has also been used to follow such processes as electron-transfer or solvation dynamics at an interface.

Nonlinear SSNLO techniques, such as SHG, have previously been confined mainly to physics and chemistry since relatively few biological samples are intrinsically non-linearly active. Examples include the use of an optically nonlinear active dye that is used to image biological cells (Campagnola et al., Peleg, 1999). In this technique, nonlinear active stains are immobilized in membranes and these stains are used to image the cell surfaces. However, the stains intercalate into the membranes in either an ‘up’ or ‘down’ direction, thus reducing the total nonlinear signal due to destructive interference. Nonlinear optically active dyes have also been used to measure the kinetics of those dyes crossing lipid bilayers in liposomes (Srivastava and Eisenthal). Recently, too, the concept and technique of second harmonic active labels (“SHG labels”) was introduced, allowing any non-linear active molecule or particle to be rendered non-linear active. The first example of this was demonstrated by labeling the protein cytochrome c with an oxazole dye and detecting the protein conjugate at an air-water interface with second harmonic generation.

### DESCRIPTION OF THE INVENTION

The present invention is based on the use of nonlinear-active labels, the surface-selectivity of second harmonic (or sum/difference frequency) generation and the fact that the nonlinear beam is scattered from an interface in a predictable, well-defined direction (in contrast to fluorescence detection in which fluorescence is emitted somewhat at random). The surface-selective nonlinear optical techniques are coherent techniques, meaning that the fundamental and nonlinear beams have well-defined phase relationships, and the wavefronts of a nonlinear beam in a macroscopic sample (within the coherence length) are in phase. These properties offer a number of advantages useful for surface or high-throughput studies in which either a single surface or a microarray surface is studied. An apparatus using nonlinear optical suface-selective-based detection, such as with second harmonic generation, requires minimal collection optics since generation of the nonlinear light only occurs at the interface and thus, in principle, allows extremely high depth discrimination and fast scanning. The probe-target interactions can be correlated with the present invention to the following measurable information, for example:

i) the intensity of the nonlinear or fundamental light.

ii) the wavelength or spectrum of the nonlinear or fundamental light.

iii) position of incidence of the fundamental light on the surface or substrate (e.g., for imaging).

iv) the time-course of i), ii) or iii).

v) one or more combinations of i), ii) and iii).

For example, probe-target binding can be measured by detecting the intensity of nonlinear optical light (e.g., second harmonic light) at some position on a substrate with surface-attached probes; the intensity of the second harmonic light changes as labelled targets (targets labelled with second-harmonic-active labels possessing a hyperpolarizability) bind to the probes at the surface and become partially oriented because of the binding, thus satisfying the non-centrosymmetric condition for generation of second harmonic light at the interface. Modeling of the intensity of light with concentration of probe-target binding complexes at the interface can be accomplished using a variety of methods, for instance by calibrating the technique for a given probe-target interaction using radiolabels or fluorescence tags.

The advantages of the present invention are enumerated as follows:

i) Detection of interfacial species in the presence of bulk species in real time. This property can be especially useful when the presence of bulk species are necessary to detect a binding process (eg., if equilibrium or real-time kinetics data is required via, for example, changing target concentrations) or the wash-away step to remove non-bound material is time-consuming, incomplete or gives artifactual results.

ii) Higher signal to noise (lower background) than fluorescence-based detection since SSNLO is generated only at non-centrosymmetric surfaces. SSNLO techniques thus have a very narrow ‘depth of field’. Sources of fluorescence in fluorescence-based detection schemes include that from materials in the field of view but not in the focal plane, autofluorescence, and contamination of the emitted fluorescence with stray excitation light; these are not sources of background nonlinear optical radiation.

iii) The technique is useful when the presence of a liquid solution is required for the measurement, i.e. where the binding process can be obviated or disturbed by a wash-away step. This aspect of the invention can be useful for equilibrium measurements (free energy, binding constants, etc.), which require the presence of bulk species or kinetics measurements with measurements made over a period of time.

iv) The scattering process responsible for the nonlinear effect in SSNLO techniques does not lead to irreversible bleaching of the label as quickly as with fluorescent labels—the two-photon absorption cross-section is much lower than the one-photon cross-section in a molecule and the NLO technique involves scattering, not absorption.

iv) A minimum of collection optics is needed and higher signal to noise is expected since the fundamental and nonlinear beams (i.e., second harmonic) have well-defined incoming and outgoing directions with respect to the interface. This is advantageous compared to fluorescence-based detection in which the fluorescence is emitted isotropically and there may be a large auto-fluorescence background out of the plane of interest (e.g., the interface containing the probes).

v) Ease of use with beads, cells or other particles whose surface makes an interface with the supporting medium, solution, etc.

### EXAMPLES OF THE USE OF THE INVENTION

Although the present invention may be used in many scientific areas of analysis and in particular, in the chemical and biological arts, the present invention can be especially useful in genomics or proteomics, where speed and ease of very high-throughput detection are critical. It may be advantageous to detect the surface species in the presence of bulk species—for instance in DNA hybridization or protein-protein detection, the wash-away step for unbound molecules would not be necessary, useful in cases where this step may contribute artifacts to the desired signal. Moreover, in many techniques, such as fluorescence-based detection, a large portion of the sample not at or near the interface (i.e., in the bulk) may contribute undesirably or interfere with measurements. It would be advantageous therefore to use a surface-selective technique such as second harmonic generation or sum frequency generation which is sensitive only to the interface.

SSNLO techniques, when used to study proteins, cells or other molecules in an array format on some surface (two-dimensional ordering of the samples on a solid surface), have other important advantages over the art. Because the technique relies on a scattering (reflection-like) process, the nonlinear beam has a well-defined direction. With fluorescence-based detection the collection optics may be complicated and extensive because the emission is isotropic and only emission from a narrow depth of field is desired. When using nonlinear optical techniques, however, the technique is intrinsically surface-selective—the ‘depth of field’ is confined by the nature of the technique to an extremely thin layer near the interface. Moreoever, the scattered light from the surface possesses a well-defined direction, so that its position at a detector can be mapped directly to a location on the array surface.

Art scanning of microarrays includes confocal-based schemes and non-confocal based schemes. U.S. Pat. No. 5,834,758 (Trulson et al.—relevant portions of which are incorporated by reference herein) describes a non-confocal based scheme for imaging a microarray using fluorescence detection. However, the sample must lie very flat in order to image only within a single focal plane for good out-of-plane discrimination. Therefore, a very finely adjustable translation stage requiring specialized components must be used for this purpose adding to the cost of the instrument and possibly the lifetime as well. The image quality of this type of apparatus can be sensitive to mechanical vibrations. Furthermore, discrimination of the out-of-plane (non-surface bound) fluorophores places a limit on the sensitivity of the technique. U.S. Pat. No. 6,134,002 (Stimson et al.—relevant portions of which are incorporated by reference herein) is an example of a confocal scanning microscope device for imaging a sample plane, i.e. a microarray. Although the confocal-based techniques have good depth discrimination, the scan rate may be low due to descanning requirements and the light throughput can be low, reducing the overall signal to noise ratio and the sensitivity of the technique.

For use with nucleic acid hybridization (oligonucleotide, polynucleotide, RNA, etc.), target oligonucleotides can be reacted with the entire surface; at the probe oligonucleotide sequences in the array (corresponding to known locations) where sequence-complementary hybridization occurs, the fundamental light would give rise to a nonlinear optical signal, or a change in the background of such a signal. This can be detected and correlated with the spatial location of the array element and hence the oligonucleotide sequence.

For example, two major applications of nucleic acid microarrays are: 1) Identification of sequence (gene or gene mutation)—monitoring of DNA variations, for example and 2) Determination of expression level (abundance) of genes. There are many formats for preparing the arrays. For example, in one case probe cDNA (500˜5000 base pairs long) can be immobilized to a solid surface such as glass using robot spotting and exposed to a set of targets either separately or in a mixture (ref. Ekins and Chu). Another format involves synthesizing oligonucleotides (20˜25 mer oligos) or peptide nucleic acids probes in-situ (on the solid substrate, Fodor et al.) or by conventional synthesis followed by on-chip immobilization. The array is then exposed to target DNA, hybridized, and the identity or abundance of complementary sequences are determined. Protein arrays can be prepared (see for example, MacBeath and Schreiber, 2000) to determine whether a given target protein binds to the immobilized probe protein on the surface. These arrays were also used to study small molecule binding to the probe proteins. Many reviews of microarray technology and applications are available in the art. For instance, those of: Ramsay (1998—relevant portions of which are incorporated by reference herein), Marshall (1998—relevant portions of which are incorporated by reference herein), Fodor (1997—relevant portions of which are incorporated by reference herein), Duggan et al. (1999—relevant (1998), Marshall (1998), Fodor (1997), Duggan et al. (1999), Schena et al. (1995), Brown et al. (1999),portions of which are incorporated by reference herein), Schena et al. (1995—relevant portions of which are incorporated by reference herein), Brown et al. (1999—relevant portions of which are incorporated by reference herein), McAllister et al. (1997) and Blanchard et al. (1996—relevant portions of which are incorporated by reference herein).

The invention can be used for studying binding processes between other biological components: cells with viruses; protein-protein interactions; protein-ligand; cell-ligand; protein-drugs, nucleic acid-drugs, cell-small molecule; cell-nucleic acid; peptide-cell, oligo or polynucleotides, virus-cell, protein-small molecule, etc. Biomimetic membranes such as phospholipid supported bilayers (eg., egg phosphatidylcholine) can also be used and are particularly useful when studies involve membrane proteins as probes.

The invention can be used for drug screening or high-throughput screening where a candidate drug is tested for its effect on probe-target binding, i.e., to reduce or enhance probe-target binding. In other cases, for example, a drug can be tested for efficacy by its ability to bind to a receptor or other molecule on the surface of a biological cell.

Other examples of the technique's use with arrays include cellular arrays, supported lipid bilayer arrays with or without membrane or attached proteins, etc. Many methods exist in the art for coupling biomolecules (eg., nucleic acid, protein and cells) to solid supports in array format. A wide degree of flexibility may be used in providing the means by which the arrays are created. They can involve, for example, covalent or non-covalent coupling to the substrate directly, to a chemically derivatized substrate, to an intermediate layer of some kind (e.g., self-assembled monolayer, a hydrogel or other bio-compatible layer known in the art). The identity of the probes (e.g., protein structure or oligonucleotide sequence) can vary from site to site across the solid surface, or the same probe can uniformly cover the surface. Targets can be of a single identity or a combination of targets with different identities. The arrays can be prepared in a variety of ways including, but not limited to, ink-jet printing, photolithography, micro-contact printing, or any other manner known to one skilled in the art of fabricating them.

Because the binding process can be measured in real time and in the presence of bulk biological components due to the surface-selectivity of the nonlinear optical technique, equilibrium binding curves and kinetics can be measured, the bulk concentration of the components can be varied, and a “wash-away” step to remove unbound components, as is used with fluorescence-based detection, may be unnecessary.

In another aspect of the invention, SHG labels—for example, as second-harmonic active molecules or particles—can be used for imaging studies of cells, membranes, tissues involving techniques such as second harmonic (or sum/difference frequency) microscopy or confocal microscopy by labeling specific probes, cell membranes, surfaces, etc. in-vitro or in-vivo. For in-vivo applications, the labels can be delivered to the sample of interest by well known techniques that use fluorescent dyes for imaging or tracing and, for example, endoscopes.

A wide degree of flexibility is expected in the design of the apparatus including, but not limited to, the source of the fundamental light, the optical train necessary to control, focus or direct the fundamental and nonlinear light beams, the design of the array, the detection system, and the use of a grating or filters and collection optics. The mode of generation (irradiation) or collection can be varied including, for example, the use of evanescent wave (total internal reflection), planar wave guide, reflection, or transmission geometries, fiber-optic, near-field illumination, confocal techniques or the use of a microcavity or integrating detection system. A number of methods for scanning a microarray on a solid surface are described. Examples include U.S. Pat. No.'s Trulson et al. (1998), Trulson et al. (2000), Stern et al. (1997) and Sampas (2000)—relevant portions of which are incorporated by reference herein.

Because the second harmonic light beam makes a definite angle to the surface plane, one can read-out the properties of the nonlinear optical radiation (for instance, as a function of fundamental incidence position in a two-dimensional array format) without needing to mechanically translate the detector or sample and without extensive collection optics. In the ‘beam scanning’ embodiment, no mechanical translation of sample surface or detector is required—only a change in a direction and/or angle of the fundamental incidence on the sample (for a fixed sample and detector)—the apparatus offers much faster scanning capability, improved ease of manufacturing and a longer lifetime.

The interface can comprise a silica, glass, silicon, polystyrene, nylon, plastic, a metal, semiconductor or insulator surface, or any surface to which biological components can adsorb or be attached. The interface can also include biological cell and liposome surfaces. The attachment or immobilization can occur through a variety of techniques well known in the art. For example, oligonucleotides can be prepared via techniques described in “Microarray Biochip Technology”, M. Schena (Ed.), Eaton Publishing, 1998—relevant portions of which are incorporated by reference herein. And, for example with proteins, the surface can be derivatized with aldehyde silanes for coupling to amines on surfaces of biomolecules (MacBeath and Schreiber, 2000—relevant portions of which are incorporated by reference herein). BSA-NHS (BSA-N-hydroxysuccinimide) surfaces can also be used by first attaching a molecular layer of BSA to the surface and then activating it with N,N′-disuccinimidyl carbonate. The activated lysine, aspartate or glutamate residues on the BSA react with surface amines on the proteins.

Supported phospholipid bilayers can also be used, with or without membrane proteins or other membrane-associated components as, for example, in Salafsky et al., Biochemistry, 1996—relevant portions of which are incorporated by reference herein, “Biomembranes”, Gennis, Springer-Verlag, Kalb et al., 1992 and Brian et al., 1984, relevant portions of which are incorporated herein. Supported phospholipid bilayers are well known in the art and there are numerous techniques available for their fabrication, with or without associated membrane proteins. These supported bilayers typically must be submerged in aqueous solution to prevent their destruction when they become exposed to air.

If a solid surface is used (e.g., planar substrate, beads, etc.) it can also be derivatized via various chemical reactions to either reduce or enhance its net surface charge density to optimize the detection of probe-target interactions (e.g., a hybridization process).

The binding process can be performed in the presence of small molecules, drugs, blocking agents, or other components which modulate the binding process.

The surface arrays can be constructed according a plurality of methods found in the art. For DNA microarrays, most are prepared with one of three non-standard approaches (Case-Green, 1998): Affymetrix, Inc. probe arrays are prepared using patterned, light-directed combinatorial chemical synthesis (Fodor, 1997); spotted arrays can be made according to Duggan (1999), Schena (1995), Brown and Botstein (1999) and McAllister (1997); ink-jet techniques can also be used to synthesize oligonucleotides base by base through sequential solution-based reactions on an appropriate substrate (Blanchard, 1996)—relevant portions of all of which references are incorporated by reference herein.

For example, nucleic acid, oligo- or nucleotide arrays can be constructed according to U.S. Pat. Nos. 6,110,426, 5,143,854 6,110,426—relevant portions of which are incorporated by reference herein, U.S. Pat. No. 5,143,854—relevant portions of which are incorporated by reference herein or Fodor (1991). Soluble protein arrays can be constructed according to Ekins (1999) relevant portions of which are incorporated by reference herein. Membrane proteins arrays can be constucted by micropatterning of fluid lipid membranes according, for example, to the method of Groves et al. (1997—relevant portions of which are incorporated by reference herein). The array substrate can be composed of glass, silicon, indium tin oxide, or any other substrate known in the art. The surface array under study can contain physical barriers between elements so that the elements (and their biomolecules) can remain in isolation from each other during a chemical reaction step. The array locations can consist of different probes, the same probes everywhere, or some combination thereof. The array can also be constructed on the underside of a prism allowing for total internal reflection of the beam and evanescent generation of the nonlinear light. Or an array substrate can be brought into contact with a prism with the same result.

An electrophoretic system can also be used in conjunction with the surface array, for example to provide reagents or biological components to one or a plurality of locations using flow channels or microcapillaries. For instance, the sample can include an array of microcapillary channels, each distinct from the other and each allowing a target-probe reaction to occur; the imaging technique would then consist of array elements, each one a microcapillary channel or reaction chamber into which the channel feeds and drains.

The polarization of the fundamental and nonlinear beams can be selected with polarizing optics elements. By analyzing the intensity of the nonlinear beam as a function of fundamental and nonlinear polarization, more information (e.g., higher signal to noise) about the probe-target complexes can be obtained. Furthermore, by selecting and analyzing the polarization of the fundamental or nonlinear optical radiation, background radiation can be reduced.

Detection can be accomplished with the use of multiple internal reflection plates (N. J. Harrick—relevant portions of which are incorporated by reference herein) allowing the fundamental beam to make multiple contacts with the array surface, thus increasing the intensity of the generated nonlinear light. Another alternative is to construct an optical cavity with the array surface on one side and a lossy coupler at one end to permit the output coupling of the nonlinear light, creating an optical microcavity which would allow the buildup of very high intensities under resonance and thus increase the amount of nonlinear light generated.

There are many linking moieties and methodologies for attaching molecules which can be nonlinear-active labels to the 5′ or 3′termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′thiol group on oligonucleotide); Sharma et al., Nucleic Acids Research, 19: 3019 (1991) (3′sulfhydryl); Giusti et al., PCR Methods and Applications, 2: 223-227 (1993) and Fung et al., U.S. Pat. No. 4,757,141 (5′phosphoamino group via Aminolink.TM.II available from Applied Biosystems, Foster City, Calif.) Stabinsky, U.S. Pat. No. 4,739,044 (3′aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′mercapto group); Nelson et al., Nucleic Acids Research, 17: 7187-7194 (1989) (3′amino group); and the like, relevant portions of which are incorporated by reference herein.

Preferably, commercially available linking moieties are employed that can be attached to an oligonucleotide during synthesis, e.g., available from Clontech Laboratories (Palo Alto, Calif.). Rhodamine and fluorescein dyes are also conveniently attached to the 5′hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928, relevant portions of which are incorporated by reference herein.

Protein arrays can be used to determine whether a given target protein binds to the immobilized probe protein on the surface; these arrays were also used to study small molecule binding to the probe proteins. Protein arrays can be prepared by the method of MacBeath and Schreiber (2000), for example, to determine whether a given target protein binds to the immobilized probe protein on the surface.

The support on which the sequences are formed may be composed from a wide range of material, either biological, nonbiological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat but may take on a variety of alternative surface configurations. For example, the substrate may contain raised or depressed regions on which a sample is located. The substrate and its surface preferably form a rigid support on which the sample can be formed. The substrate and its surface are also chosen to provide appropriate light-absorbing characteristics. For instance, the substrate may be a polymerized Langmuir Blodgett film, functionalized glass, Si, Ge, GaAs, GaP, SiO.sub.2, SiN.sub.4, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art upon review of this disclosure. In a preferred embodiment the substrate is flat glass or silica.

According to some embodiments, the surface of the substrate is etched using well known techniques to provide for desired surface features. For example, by way of the formation of trenches, v-grooves, mesa structures, or the like, the synthesis regions may be more closely placed within the focus point of impinging light. The surface may also be provided with reflective “mirror” structures for maximization of emission collected therefrom.

The identity of the probes (e.g., protein structure or oligonucleotide sequence) can vary from site to site across the solid surface, or the same probe can uniformly cover the surface. Targets can be of a single identity or a combination of targets with different identities.

In another aspect of the invention, labels can be attached to the surface of a cell or liposome containing ion channel proteins. The nonlinear properties (e.g., hyperpolarizability) of the labels is sensitive to the surface electric potential of the cells or liposomes. When the ion channels open or close or their properties are otherwise changed, the surface electric potential of the cells or liposomes can change in turn, thus changing the nonlinear properties of the labels and in turn the detected nonlinear radiation. Thus, this aspect of the invention can be used, for example, to detect ligand binding to ion channel receptors (or to other receptors which will trigger an ion channel behavior). The binding can be monitored in the presence of drugs, agonists, antagonists, etc., and in real-time if desired.

Proteins can be immobilized to a solid surface. For example, they can be attached using the methods of MacBeath and Schreiber (Science, 2000). For example, protein G molecules can be immobilized to a derivatized surface via the method of MacBeath and Schreiber. These are the probes. Immunoglobulin G (IgG) proteins are used as the targets and a solution of IgG is brought into contact with the protein G surface. Protein G molecules should possess a net orientation at the interface if possible. Decorators are anti-IgG proteins which have been previously labeled with “SHG labels”, rendering them detectable via second harmonic generation. The labels can be oxazole dye (Salafsky et al., 2000) or a non-centrosymmetric Au particle with long linkers. As IgG targets bind to protein G probes, the amount of IgG at the interface increases and so does the SHG signal intensity since the number of SHG-labels at the interface increases. The SHG-labels on the anti-IgG can be attached via linkers to maximize their orientation when bound to targets. Even if the targets are randomly oriented within the population of target-probe complexes on the surface, the labels can be non-randomly oriented using linkers or spacer molecules, and this will ensure their detection via second harmonic generation. Decorators not associated with the interfacial targets will be isotropically oriented and will not produce a second harmonic signal. This signal can be quantitatively modeled using a Langmuir adsorption curve to determine the concentration of probe-target complexes using software and a PC as described for cytochrome c adsorption to silica in Salafsky, 2000. Relevant portions of the aforementioned references are incorporated by references herein.

Applications of the labels include studies of protein-protein binding at an interface, protein or virus binding to a cell surface, oligonucleotide hybridization at a solid interface—such as with microarrays—two-photon absorption studies, two-photon microscopy, nonlinear optical microscopy (eg., SHG microscopy), cell sorting using a nonlinear optical technique, drug-receptor interaction, etc.

One means of determining whether a particular molecule or particle is a candidate for use as a nonlinear-active label is by studying it using second harmonic generation at an air-water interface. For instance, in the case of particles, if the particles assemble at the air-water interface in a manner which gives a net orientation of the particles (on a length scale of the coherence length) the layer of particles will generate second harmonic light. Another means of doing this is by measuring a sample of a suspension of the particles and detecting the hyper-rayleigh scattering. Yet another means involves the use of EFISH (Electric-field induced second harmonic generation). EFISH can be used to determine if a candidate molecule or particle is nonlinearly active. Electric field induced second harmonic (EFISH) is well known in the field of nonlinear optics. This is a third order nonlinear optical effect, with the polarization source written as: P⁽²⁾(ω₃)=χ⁽²⁾(−ω₃; ω₁,ω₂): E^{ω1} E^{ω2}. The effect can be used to measure the hyperpolarizabilty of molecules in solution by using a dc field to induce alignment in the medium, and allowing SHG to be observed. This type of measurement does not require that the particle themselves be ordered at an interface, but does require that the particles be nonlinear active and thus non-centrosymmetric.

Examples of samples in which the labels can be of use include, but are not limited to, solid surfaces with immobilized protein, oligonucleotides or cells and supported phospholipid bilayers. The surface geometry can be varied, indeed spherical beads and other non-planar geometries are generally accessible with the nonlinear optical techniques.

In one important aspect of the invention, the use of the linkers which couple the labels to their targets can be made long enough so that the orientation of the targets at the interface (i.e., when bound to the probes) does not significantly effect the orientation of the label. Because the intensity of the nonlinear light generated will depend on the net orientation of the labels at the interface—and the orientation of the targets at an interface can be difficult to control (i.e., the targets may even be randomly oriented at the interface)—the use of linkers can separate the labels sufficiently from the targets so that the orientation of the latter does not necessarily determine the orientation of the former. In cases where this is less important, for example, with integral membrane proteins in supported lipid bilayers on glass, where the orientation of the membrane protein presented to the targets is generally uniform, this aspect of the linkers can be less important. Nevertheless, in most cases, linkers may still be necessary in order to couple the label to the targets.

Cells bound to a substrate can also be used to determine protein-cell binding, virus-cell binding, etc. where the cell is the probe component and proteins, viruses, etc. are the target components. The next section discusses the well known art for coupling cells to solid substrates.

Various art not involving the use of a surface-selective nonlinear optical technique contains relevant portions for the present invention and the following exemplary list and their references therein is referenced herein: King et al., U.S. Pat. No. 5,633,724 for the scanning and analysis of the scans; Fork et al., U.S. Pat. No. 6,121,983 for the multiplexing of a laser to produce a laser array suitable for scanning; Foster, U.S. Pat. No. 5,485,277; Fodor et al., U.S. Pat. No. 5,324,633 and Fodor et al., U.S. Pat. No. 6,124,102 for a substrate containing an array of attached probes and for the analysis of scans to determine kinetic and equilibrium properties of a binding reaction between probes and targets; Kain et al., U.S. Pat. No. 5,847,400 for laser scanning of a substrate; King et al., U.S. Pat. No. 5,432,610 for an optical resonance cavity for power build-up; Walt et al., U.S. Pat. No. 5,320,814, Walt et al., U.S. Pat. No. 5,250,264, Walt et al., U.S. Pat. No. 5,298,741, Walt et al., U.S. Pat. No. 5,252,494, Walt et al., U.S. Pat. No. 6,023,540, Walt et al., U.S. Pat. No. 5,814,524, Walt et al., U.S. Pat. No. 5,244,813 for fiber-optic-based apparatus; Fiekowsky et al., U.S. Pat. No. 6,095,555 for imaging and software-based analysis of images; Stem et al., U.S. Pat. No. 5,631,734 for data acquisition; Stimson et al., U.S. Pat. No. 6,134,002 for confocal imaging techniques; Sampas, U.S. Pat. No. 6,084,991 for CCD-based imaging techniques; Stem et al., U.S. Pat. No. 5,631,734 for photolithographical preparation of probes attached to surfaces; Shalon et al., U.S. Pat. No. 6,110,426 for methods and apparatus for creating attached probes on a surface; Slettnes, U.S. Pat. No. 6,040,586 for position-based scanning techniques; Trulson et al, U.S. Pat. No. 6,025,601 for methods of imaging probe-target binding on a surface.

Microarrays of Cells

This section outlines some of the methods concerned with fabricating arrays of biological cells on surfaces, one type of array amenable to study using the present invention. Many methods have been described for making uniform micro-patterned arrays of cells for other applications, using for example photochemical resist-photolithograpy. (Mrksich and Whitesides, Ann. Rev. Biophys. Biomol. Struct. 25:55-78, 1996). According to this photoresist method, a glass plate is uniformly coated with a photoresist and a photo mask is placed over the photoresist coating to define the “array” or pattern desired. Upon exposure to light, the photoresist in the unmasked areas is removed. The entire photolithographically defined surface is uniformly coated with a hydrophobic substance such as an organosilane that binds both to the areas of exposed glass and the areas covered with the photoresist. The photoresist is then stripped from the glass surface, exposing an array of spots of exposed glass. The glass plate then is washed with an organosilane having terminal hydrophilic groups or chemically reactable groups such as amino groups. The hydrophobic organosilane binds to the spots of exposed glass with the resulting glass plate having an array of hydrophilic or reactable spots (located in the areas of the original photoresist) across a hydrophobic surface. The array of spots of hydrophilic groups provides a substrate for non-specific and non-covalent binding of certain cells, including those of neuronal origin (Klienfeld et al., J. Neurosci. 8:4098-4120, 1988). Reactive ion etching has been similarly used on the surface of silicon wafers to produce surfaces patterned with two different types of texture (Craighead et al., Appl. Phys. Lett. 37:653, 1980; Craighead et al., J. Vac. Sci. Technol. 20:316, 1982; Suh et al. Proc. SPIE 382:199, 1983).

In another method based on specific yet non-covalent interactions, photoresist stamping is used to produce a gold surface coated with protein adsorptive alkanethiol. (Singhvi et al., Science 264:696-698, 1994). The bare gold surface is then coated with polyethylene-terminated alkanethiols that resist protein adsorption. After exposure of the entire surface to laminin, a cell-binding protein found in the extracellular matrix, living hepatocytes attach uniformly to, and grow upon, the laminin coated islands (Singhvi et al. 1994). An elaboration involving strong, but non-covalent, metal chelation has been used to coat gold surfaces with patterns of specific proteins (Sigal et al., Anal. Chem. 68:490-497, 1996). In this case, the gold surface is patterned with alkanethiols terminated with nitriloacetic acid. Bare regions of gold are coated with tri(ethyleneglycol) to reduce protein adsorption. After adding Ni²⁺, the specific adsorption of five histidine-tagged proteins is found to be kinetically stable.

More specific uniform cell-binding can be achieved by chemically crosslinking specific molecules, such as proteins, to reactable sites on the patterned substrate. (Aplin and Hughes, Analyt. Biochem. 113:144-148, 1981). Another elaboration of substrate patterning optically creates an array of reactable spots. A glass plate is washed with an organosilane that chemisorbs to the glass to coat the glass. The organosilane coating is irradiated by deep UV light through an optical mask that defines a pattern of an array. The irradiation cleaves the Si—C bond to form a reactive Si radical. Reaction with water causes the Si radicals to form polar silanol groups. The polar silanol groups constitute spots on the array and are further modified to couple other reactable molecules to the spots, as disclosed in U.S. Pat. No. 5,324,591, incorporated by reference herein. For example, a silane containing a biologically functional group such as a free amino moiety can be reacted with the silanol groups. The free amino groups can then be used as sites of covalent attachment for biomolecules such as proteins, nucleic acids, carbohydrates, and lipids. Other methods for patterning the adhesion of mammalian cells to surfaces using self-assembled monolayers on a surface include Lopez et al. 1993 and Stenger et al., 1992.

The non-patterned covalent attachment of a lectin, known to interact with the surface of cells, to a glass substrate through reactive amino groups has been demonstrated (Aplin & Hughes, 1981). The optical method of forming a uniform array of cells on a support requires fewer steps and is faster than the photoresist method, (i.e., only two steps), but it requires the use of high intensity ultraviolet light from an expensive light source.

In all of these methods the resulting array of cells is uniform, since the biochemically specific molecules are bound to the micro-patterned chemical array uniformly. In the photoresist method, cells bind to the array of hydrophilic spots and/or specific molecules attached to the spots which, in turn, bind cells. Thus cells bind to all spots in the array in the same manner. In the optical method, cells bind to the array of spots of free amino groups by adhesion. Methods for attaching a variety of cell types to the same substrate for simultaneously binding against these cell types also exist.

Peptide-nucleic Acids

In an alternative embodiment, peptide nucleic acids or oligomers, which are analogs of nucleic acids in which, for example, the peptide-like backbone is replaced with an uncharged backbone, can be used with the present invention. PNAs are well known in the art. References below give extensive reviews of the use of these nucleic acid analogs in a wide range of applications, including surface and array-based hybridization wherein PNAs are attached to surfaces and allowed to bind with sequence-complementary DNAs or RNAs.

For instance, oligomers of PNA can be used as the surface-attached probe components instead of DNA oligomers. A key advantage to using PNAs is that the hybridization reaction with DNAs or RNAs, for example, (containing charged phosphate groups) is only weakly dependent (eg., the melting temperature) on ionic strength because there is much less charge repulsion as found with conventional DNA-DNA, etc. hybridization. Thus, one can use the surface-selective nonlinear optical technique to follow a probe-target hybridization at any desired ionic strength.

The PNAs are commercially available (for instance via Applied Biosystems, Foster City, Calif.) or other analogs of DNA can be synthesized and used.

The following references are broad reviews of the use of PNAs.

Nielsen, et al. “Peptide nucleic acids (PNA): Oligonucleotide analogues with a polyamide backbone” Antisense Research and Applications (1992) 363-372

Nielsen, et al. “Peptide nucleic acids (PNAs): Potential Antisense and Anti-gene Agents.” Anti-Cancer Drug Design 8 (1993) 53-63

Buchardt, et al. “Peptide nucleic acids and their potential applications in biotechnology” TIBTECH 11 (1993) 384-386

Nielsen, P. E., Egholm, M. and Buchardt, O. “Peptide Nucleic Acid (PNA). A DNA mimic with a peptide backbone” Bioconjugate Chemistry 5 (1994) 3-7

Nielsen “Peptide nucleic acid (PNA): A lead for gene therapeutic drugs” Antisense Therapeutics 4 (1996) 76-84

Nielsen, P. E. “DNA analogues with nonphosphodiester backbones” Annu.Rev.Biophys.Biomol.Struct. 24 (1995) 167-183

Hyrup, B. and Nielsen, P. E. “Peptide Nucleic Acids (PNA): Synthesis, Properties and Potential Applications” Bioorg. Med. 4 (1996) 5-23

Mesmaeker, A. D., Altman, K.-H., Waldner, A. and Wendeborn, S. “Backbone modifications in oligonucleotides and peptide nucleic acid systems” Curr. Opin. Struct. Biol. 5 (1995) 343-355

Noble, et al. “Impact on Biophysical Parameters on the Biological Assessment of Peptide Nucleic Acids, Antisense Inhibitors of Gene Expression” Drug.Develop.Res. 34 (1995) 184-195

Dueholm, K. L. and Nielsen, P. E. “Chemistry, properties, and applications of PNA (Peptide Nucleic Acid)” New J. Chem. 21 (1997) 19-31

Knudsen and Nielsen “Application of Peptide Nucleic Acid in Cancer Therapy” Anti-Cancer Drug 8 (1997) 113-118

Nielsen, P. E. “Design of Sequence-Specific DNA-Binding Ligands” Chem. Eur. J. 3 (1997) 505-508

Corey “Peptide nucleic acids: expanding the scope of nucleic acid recognition” TIBTECH 15 (1997) 224-229

Nielsen, P. E. and Ørum, H. “Peptide nucleic acid (PNA), a new molecular tool.” In Molecular Biology: Current Innovations and Future Trends, Part2. Horizon Scientific Press, (1995) 73-89

Nielsen, P. E. and Haaima, G. “Peptide nucleic acid (PNA). A DNA mimic with a pseudopeptide backbone” Chem. Soc. Rev. (1997) 73-78

Ørum, H., Kessler, C. and Koch, T. “Peptide Nucleic Acid” Nucleic Acid Amplification Technologies: Application to Disease Diagnostics (1997) 29-48

Wittung, P., Nielsen, P. and Norden, B. “Recognition of double-stranded DNA by peptide nucleic acid” Nucleosid. Nucleotid. 16 (1997) 599-602

Weisz, K. “Polyamides as artificial regulators of gene expression” Angew. Chem. Int. Ed. Eng 36 (1997) 2592-2594

Nielsen, P. E. “Structural and Biological Properties of Peptide Nucleic Acid (Pna)” Pure & Applied Chemistry 70 (1998) 105-110

Nielsen, P. E. “Sequence-specific recognition of double-stranded DNA by peptide nucleic acids” Advances in DNA Sequence-Specific Agents 3 (1998) 267-278

Nielsen “Antisense Properties of Peptide Nucleic Acid” Handbook of Experimental Pharmacology 131 (1998) 545-560

Nielsen “Peptide Nucleic Acids” Science and Medicine (1998) 48-55

Uhlmann, E. “Peptide nucleic acids (PNA) and PNA-DNA chimeras: from high binding affinity towards biological function” Biol Chem 379 (1998) 1045-52

Wang “DNA biosensors based on peptide nucleic acid (PNA) recognition layers. A review” Biosens Bioelectron 13 (1998) 757-62

Uhlmann, E., Peyman, A., Breipohl, G. and Will, D. W. “PNA: Synthetic polyamide nucleic acids with unusual binding properties” Angewandte Chemie-International Edition 37 (1998) 2797-2823

Nielsen, P. E. “Applications of peptide nucleic acids” Curr Opin Biotechnol 10 (1999) 71-75

Bakhtiar, R. “Peptide nucleic acids: deoxyribonucleic acid mimics with a peptide backbone” Biochem. Educ. 26 (1998) 277-280

Lazurkin, Y. S. “Stability and specificity of triplexes formed by peptide nucleic acid with DNA” Mol. Biol. 33 (1999) 79-83

Nielsen and Egholm “Peptide Nucleic Acids: Protocols and Applications” (1999) 266 pp.

Eldrup and Nielsen “Peptide nucleic acids: potential as antisense and antigene drugs” Adv. Amino Acid Mimetics Peptidomimetics 2 (1999) 221-245

Bentin, T. and Nielsen, P. E. “Triplexes involving PNA” Triple Helix Form. Oligonucleotides (1999) 245-255

Falkiewicz, B. “Peptide nucleic acids and their structural modifications” Acta Biochim. Pol. 46 (1999) 509-529.

The following references are descriptions of the use of PNAs in array-based detection, including means for attaching the PNA probes to the solid surface.

Hoffmann, R., et al. “Low scale multiple array synthesis and DNA hybridization of peptide nucleic acids” Pept. Proc. Am. Pept. Symp., 15th (1999) 233-234

Matysiak, S., Hauser, N. C., Wurtz, S. and Hoheisel, J. D. “Improved solid supports and spacer/linker systems for the synthesis of spatially addressable PNA-libraries” Nucleosides Nucleotides 18 (1999) 1289-1291.

Decorators

In another aspect of the present invention, a “decorator” molecule or particle is used to detect probe-target binding reactions. A decorator molecule or particle will possess a hyperpolarizability and can be used to reveal probe-target binding interactions via a surface-selective nonlinear optical technique (e.g., second harmonic generation) through the specific binding affinity it will have for the targets, the probes or the target-probe complex, or other species which recognize the targets, the probes or the target-probe-complex. The technique is useful when probe or targets are not appreciably nonlinear optical active (e.g., do not possess a hyperpolarizability). Decorators can intrinsically possess a hyperpolarizability or be themselves labeled with a moiety which is nonlinear-optically active (e.g., second harmonic active). Decorators can be present during the probe-target binding process, or added afterwards to reveal the sites where binding has occurred. The decorator molecule or particle can be dissolved or suspended in the solution or aqueous phase containing the target components—and it should not appreciably alter or participate in the target-probe reaction.

An example of the invention is the case of proteins immobilized to a solid substrate, either in a microarray or patterned form, or uniformly across a surface—and with protein composition either varying or the same from site to site on the surface. At a given site, site A, protein P (the probe) will be immobilized. Protein K (the target) binds to protein P to form their complex, KP. Also, a decorator protein —Q— with an “SHG” label attached to it, has a specific binding affinity for protein K. One can introduce the substrate with immobilized proteins P to a solution containing the targets (K); without K bound to the surface, there is a small background SHG signal present. As K binds to P, the amount of the decorator Q (and the SHG label) at the solid surface (and partially oriented by it) will increase, leading to an increase in the SHG optical signal intensity. The same type of measurement can also be made in the presence of drugs, antagonists, agonists, or any other compounds which modulate the K—P binding reaction (for example, the equilibrium constant). The measurements can be made in real-time if necessary. Furthermore, the decorator Q can be added to the solution some time after K has been introduced to the surface containing the probes P.

Another important use of the invention is in detection of DNA or other nucleic acid or analog binding. A single stranded probe is immobilized to a surface, a microsphere bead at the distal end of a fiber optic, for example. One is interested in probing a pool of unknown or known strands for the amount of sequence-complementary targets for a given probe sequence. The probe and target strands are single stranded, while their bound complex is double stranded. An nonlinear-active (e.g., second-harmonic active) decorator can be used in this case which intercalates within the DNA, electrostatically binds to the backbone phosphates, or both. For example, an SH-active intercalator which can discriminate in its intercalation binding between single and double-stranded DNA will produce the desired affect: when a complementary target binds to a probe, the amount of SH-active intercalator at the sold surface will increase, leading to an increase in the optical SH signal. In another example, an SH-active decorator will bind electrostatically to either single or double stranded DNA—the number of decorators at the surface for the bound complex will be greater than the number for the single stranded probe, since there will be approximately twice as many phosphate groups available for interaction with the decorator with the double stranded probe-target complex. The decorator can be comprised of a single moiety which possesses both nonlinear optical activity such as being second harmonic active and can interact specifically (has an affinity) with the nucleic acids, for example through intercalation, electrostatic interaction, etc. Or, the decorator can be comprised of two or more moieties in which one part is SH-active and the other part possesses an affinity for the nucleic acids.

For example, well known molecules which can intercalate or electrostatically bind to DNA, or both, are as follows:

Psoralen

Ethidium bromide

Methanphosphonate

Phosophoramidites

Propidium iodide

Acridine

Acridine orange

9-amino acridine

Succinimidyl acridine-9-carboxylate

Cloroquine

Pyrine

Echinomycin

4′,6-diamidino-2-phenylindole, dihydrochloride (DAPI)

Single-strand binding protein (SSB)

Tripyrrole peptides

Flavopiridol

Pyronin Y

Hoechst 33258

Bisbenzimide

This list is illustrative and is not intended to be limiting in scope. SH-active moieties can be linked, covalently bound or otherwise bonded to, by well-known means available to one skilled in the art of synthetic organic chemistry, to any of the above listed compounds to produce a decorator compound which has both specificity for nucleic acids and a nonlinear optical activity. It is also desirable to use a decorator which is not intrinsically fluorescent, either due to the SH-active moiety or the nucleic-acid affinity moiety.

### DEFINITIONS

The following terms used throughout the present specification are intended to have the following general definitions:

1. Complementary: Refers to the topological and chemical compatibility of interacting surfaces between two biological components, such as with a ligand molecule and its receptor (also referred to sometimes in the art as ‘molecular recognition’). Thus, the receptor and its ligand can be described as complementary, and, furthermore, the contacts' surface characteristics are complementary to each other.

2. Biological (Components): This term includes any naturally occurring or modified particles or molecules found in biology, or those molecules and particles which are employed in a biological study, including probes and targets. Examples of these include, but are not limited to, a biological cell, protein, nucleic acids, antibodies, receptors, peptides, small molecules, oligonucleotides, carbohydrates, lipids, liposomes, polynucleotides and others such as drugs, toxins and genetically engineered protein or peptide.

3. Ligand: A ligand is a molecule that is recognized by a particular receptor. Examples of ligands that can be used with the present invention include, but are not restricted to, antagonists or agonists for cell membrane receptors, toxins and venoms, viral epitopes, hormones, hormone receptors, peptides, enzymes, enzyme substrates, cofactors, drugs (e.g. opiates, steroides, etc.), lectins, sugars, oligonucleotides, nucleic acids, oligosaccharides, proteins, and monoclonal antibodies.

4. Receptor: A molecule that has a chemical affinity for a given ligand. Receptors can be naturally occurring or man-made molecules. Also, they can be used in an unaltered state or as aggregates with other biological components. Receptors can be attached, covalently or noncovalently, to a binding partner, either directly or via a specific binding substance. Examples of receptors which can be employed by this invention include, but are not limited to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes and organelles. Receptors are occasionally referred to in the art as anti-ligand. As the term receptors is used herein, no difference in meaning is intended. A “Ligand Receptor Pair” is formed when two macromolecules have combined through molecular recognition to form a complex.

Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention can be useful in investigating the ligand-binding site on the antibody molecule which combines with the epitope of an antigen of interest; determining a sequence that mimics an antigenic epitope can lead to the development of vaccines of which the immunogen is based on one or more of such sequences or lead to the development of related diagnostic agents or compounds useful in therapeutic treatments such as for autoimmune diseases (e.g., by blocking the binding of the “self” antibodies).

d) Nucleic Acids: Sequences of nucleic acids can be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant. Catalytic polypeptides are described in, for example, U.S. Pat. No. 5,215,899, which is incorporated herein by reference for all purposes.

f) Hormone receptors: Examples of hormone receptors include, e.g., the receptors for insulin and growth hormone. Determination of the ligands which bind with high affinity to a receptor is useful in the development of, for example, an oral replacement of the daily injections which diabetics must take to relieve the symptoms of diabetes, and in the other case, a replacement for the scarce human growth hormone which can only be obtained from cadavers or by recombinant DNA technology. Other examples are the vasoconstrictive hormone receptors; determination of those ligands which bind to a receptor can lead to the development of drugs to control blood pressure.

g) Opiate receptors: Determination of ligands which bind to the opiate receptors in the brain is useful in the development of less-addictive replacements for morphine and related drugs.

h) Ion channel proteins or receptors, or cells containing ion channel receptors.

5. Surface-selective: This term refers to a non-linear optical technique such as second harmonic generation or sum/difference frequency generation in which, by symmetry, only a non-centrosymmetric surface (comprising array, substrate, solution, biological components, etc.), is capable of generating non-linear light.

6. Array or Microarray: Refers to a substrate or solid support on which is fabricated one type, or a plurality of types, of biological components in one or a plurality of known locations. This includes, but is not restricted to, two-dimensional microarrays and other patterned samples. Other terms in the art which are often used interchangeably for ‘array’ include: gene chip, gene array, biochip, DNA chip, protein chip and microarray, the latter being an array with elements of the array (patterned areas with attached probes) whose dimensions are on the order of microns.

7. Label: Refers to a nonlinear-active moiety, particle or molecule which can be attached (covalently or non-covalently) to a molecule, particle or phase (e.g., lipid bilayer) in order to render the latter more nonlinear optical active. The labels are pre-attached to the molecules or particles and unbound or unreacted labels separated from the labeled entities before a measurement is made. EFISH (Electric-field induced second harmonic generation) or Hyper-Rayleigh scattering can be used to determine if a candidate molecule or particle is nonlinearly active. Electric field induced second harmonic (EFISH) is well known in the field of nonlinear optics. This is a third order nonlinear optical effect, with the polarization source written as: P⁽²⁾(ω₃)=χ⁽²⁾(−ω₃; ω₁,ω₂): E^{ω1} E^{ω2}. The effect can be used to measure the hyperpolarizabilty of molecules in solution by using a dc field to induce alignment in the medium, and allowing SHG to be observed. This is sometimes called the reorientational mechanism.

8. Linker: A molecule which serves to chemically link (usually via covalent bonds) two different objects together. Herein a linker can be used to couple targets to non-linear active particles or moieties, targets to nonlinear-active derivatized particles, surface layers to targets, surface layers to nonlinear-active particle or moieties, etc. A linker can, for example, be a homobifunctional or heterobifunctional cross-linker molecule, a biotin-streptavidin couple wherein the biotin is attached to one of the two objects and the streptavidin to the other, etc. Many linkers are available commercially, for example from Pierce Chemical Inc., Sigma-Aldrich, Fluka, etc. In some art, the term ‘tether’, ‘spacer’ or ‘cross-linker’ is also used with the same meaning.

9. Elements: When used with ‘array’ or ‘microarray’, the meaning is a specific location among the plurality of locations on the array surface. Each element is a discrete region of finite area formed on the surface of a solid support or substrate.

10. Nonlinear: Refers herein to those optical techniques capable of transforming the frequency of an incident light beam (called the fundamental). The nonlinear beams are the higher order frequency beams which result from such a transformation, e.g. second harmonic, etc. In second harmonic, sum frequency or difference frequency generation, the nonlinear beams are generated coherently. In second harmonic generation (SHG), two photons of the fundamental beam are virtually scattered by the interface to produce one photon of the second harmonic. Also referred to herein as nonlinear optical or surface-selective nonlinear (optical) or by various combinations thereof.

11. Probe: Refers herein to biological components (eg., cells, proteins, virus, ligand, small molecule, drugs, oligonucleotides, DNA, RNA, cDNA, etc.) which are attached to a surface (e.g., solid substrate, cell surface, liposome surface, etc.), or are cells, lipsomes, particles, beads or other components which comprise a surface e.g. freely suspended in some medium in a sample cell. (In some literature in the art, this term refers to the free components which are tested for binding against the probes).

12. Target: Refers herein to biological components which are unbound to the probes' surface or surfaces comprising attached probes, and which may bind to probes.

13. Attached (Attach): Refers herein to biological components which are either prepared or engineered in-vitro to be attached to some surface, via covalent or non-covalent means, including for example the use of linker molecules to, for example, a solid substrate, a cell surface, a liposome surface, a gel substrate, etc.; or the probes are found naturally ‘attached’ to a surface such as in the example of native membrane receptors embedded in cell membranes, tissues, organs (in-vitro or in-vivo). In some instances herein, the word ‘attached’ or ‘attach’ refers also to the chemical or physical attachment of a label to a target or decorator. Also referred to herein as ‘surface-attached’.

14. Centrosymmetric: A molecule or material phase is centrosymmetric if there exists a point in space (the ‘center’ or ‘inversion center’) through which an inversion (x,y,z)→(−x,−y,−z) of all atoms is performed that leaves the molecule or material unchanged. A non-centrosymmetric molecule or material lacks this center of inversion. For example, if the molecule is of uniform composition and spherical or cubic in shape, it is centrosymmetric. Centrosymmetric molecules or materials have no nonlinear susceptibility or hyperpolarizability, necessary for second harmonic, sum frequency and difference frequency generation.

15. Nucleic Acid Analog: A non-natural nucleic acid which can function as a natural nucleic acid in some way. For example, a Peptide Nucleic Acid (PNA) is a non-natural nucleic acid because it has a peptide-like backbone rather than the phosphate background of natural nucleic acids. The PNAs can hybridize to natural nucleic acids via base-pair interactions. Another example of a Nucleic acid analog can be one in which the base pairs are non-natural in some way.

16. Decorator: Refers to a nonlinear active molecule or particle (possesses a hyperpolarizability) which can be bound to targets, probes or target-probe complexes in order to allow the detection and discrimination between them. A decorator should not appreciably alter or participate in the target-probe reaction itself. The decorator can be dissolved or suspended in the solution or aqueous phase containing the target component. A decorator is distinguished from an SH-active label (J. S. Salafsky, co-pending application ‘SH-labels . . . ’) for its specific binding affinity for targets, probes, or the target-probe complex. In the art (J. S. Salafsky K. B. Eisenthal, co-pending application ‘SHG labels . . . ’), an SHG-label is attached to a biological component—via specific chemical bonds or non-specific (e.g., electrostatic) means—and then used to follow that component to an interface. A decorator can be used to detect probe-target complexes by its specific binding affinity (in other art, ‘molecular recognition’ to the targets, probes or the target-probe complexes.

17. Binding Affinity or Affinity: The specific physico-chemical interactions between binding partners, such as a probe and target, which lead to a binding complex (affinity) between them. The binding reaction is characterized by an equilibrium constant which is a measure of the energetic strength of binding between the partners. Specificity in a binding reaction implies that probe-target binding only occurs appreciably with specific binding partners—not any at random. For example, the protein Immunoglobulin G (IgG) has a specific binding affinity for protein G and less or none for other proteins. In some art, the term ‘molecular recognition’ is used to describe the binding affinity between components.

18. Electrically Charged or Electric Charge: Defined herein as net electric charge on a particle or molecule, which confers a mobility (velocity) of said particle or molecule in an electric field. The net charge could be part of a molecular moiety such as phosphate group on nucleic acid backbones, side-chains of amino acid residues in proteins, lipid head groups in membrane lipids or cellular membranes, etc. The charge can be positive or negative and would determine the direction of mobility of the particle or molecule if said particle or molecule is placed in an electric field of a given orientation (direction of positive to negative electric potential). The charge can be non-integer multiples of the fundamental unit of charge (q≈1.6×10⁻¹⁹ C) or a fraction of the fundamental unit of charge—so-called ‘partial charges’, well known to those skilled in the art.

19. Dipolar: Defined herein as possessing an electric dipole or ‘dipole moment’ on a particle or molecule, which takes the standard definition known to one skilled in the art: the sum of all vectors μ=Q·R where Q is the amount of charge (positive or negative) at a particular spatial location (x,y,z in Cartesian coordinates) in the particle or molecule and R is the vector which points from an origin of reference (x,y,z) to the net charge Q. If the sum of these vectors results in a vector with a non-zero trace (sum of x,y,z components of the resultant vector), the particle or molecule possesses a dipole moment and is electrically dipolar.

20. Electrically Neutral: Defined herein as zero net (sum of positive and negative) electric charge on a particle or molecule, which would result in no appreciable mobility (velocity) of said particle or molecule in an electric field.

21. Hyperpolarizability or Nonlinear Susceptibility: The properties of a molecule, particle, interface or phase which allow for generation of the nonlinear light. Typical equations describing the nonlinear interaction for second harmonic generation are: α⁽²⁾(2ω)=β:E(ω)·E(ω) or P⁽²⁾(2ω)=χ⁽²⁾:E(ω)E(ω) where α and P are, respectively, the induced molecular and macroscopic dipoles oscillating at frequency 2ω, β and χ⁽²⁾ are, respectively, the hyperpolarizability and second-harmonic (nonlinear) susceptibility tensors, and E(ω) is the electric field component of the incident radiation oscillating at frequency ω. The macroscopic nonlinear susceptibility χ⁽²⁾ is related by an orientational average of the microscopic β hyperpolarizability. For sum or difference frequency generation, the driving electric fields (fundamentals) oscillate at different frequencies (i.e., ω₁ and ω₂) and the nonlinear radiation oscillates at the sum or difference frequency (ω₁±ω₂). The terms hyperpolarizability, second-order nonlinear polarizability and nonlinear susceptibility are sometimes used interchangeably, although the latter term generally refers to the macroscopic nonlinear-activity of a material or chemical phase or interface. The terms ‘nonlinear active’ or ‘nonlinearly active’ used herein also refer to the general property of the ability of molecules, particles, an interface or a phase, to generate nonlinear optical radiation when driven by incident radiation beam or beams.

22. Polarization: The net dipole per unit volume (or area) in a region of space. The polarization can be time-dependent or stationary. Polarization is defined as: ∫μ(R) dR where an integration of the net dipole is made over all volume elements in space dR near an interface.

23. Radiation: Refers herein to electromagnetic radiation or light, including the fundamental beams used to generate the nonlinear optical effect, or the nonlinear optical beams which are generated by the fundamental. Also referred to herein as ‘waves’, ‘signal’ or ‘nonlinear signal’, ‘beams’, ‘light’.

24. Near-field techniques: Those techniques known in the art to be capable of measuring or imaging optical radiation on a surface or substrate with a lateral resolution at or smaller than the diffraction-limited distance. Examples of near-field techniques (or near-field imaging) include NSOM (near-field scanning optical microscopy), whereby optical radiation (from fluorescence, second harmonic generation, etc.) is collected at a point very near the surface.

25. Detecting, Detection: When referring herein to nonlinear optical methods, refers to those techniques by which the properties of surface-selective nonlinear optical radiation can be used to detect, measure or correlate properties of probe-target interactions, or effects of the interactions, with properties of the nonlinear optical light (e.g., intensity, wavelength, polarization or other property common to electromagnetic radiation).

26. Interface: For the purpose of this invention, the interface can be defined as a region which generates a nonlinear optical signal or the region near a surface in which there are nonlinear-active labeled targets possessing a net orientation. An interface can also be composed of two surfaces, a surface in contact with a different medium (e.g., a glass surface in contact with an aqueous solution, a cell surface in contact with a buffer), the region near the contact between two media of different physical or chemical properties, etc.

27. Conjugated, Coupled: Refers herein to the state in which one particle, moiety or molecule is chemically bonded, covalently or non-covalently linked or by some means attached to a second particle moiety, molecule, surface or substrate. These means of attachment can be via electrostatic forces, covalent bonds, non-covalent bonds, physisorption, chemisorption, hydrogen bonds, van der Waal's forces or any other force which holds the probes with a binding energy to the substrate (a corallery to this definition is that some force is required to separate the probes held by the substrate from the substrate).

28. Reactions: Refers herein to chemical, physical or biological reactions including, but not limited to, the following: probes, targets, inhibitors, small molecules, drugs, antagonists, antibodies, etc. The term ‘effects of reactions’ or ‘effects of said reactions’ refers herein to physical or chemical effects of the probe-target reactions: for example, the probe-target reactions can comprise a ligand-receptor binding reaction which leads, in turn, to an ion channel opening and a change in the surface charge density of a cell, the latter being then detected by the nonlinear optical technique. The effects of the probe-target reactions, or the probe-target reactions themselves, might be referred in some art as a ‘second messenger’ reaction. Also referred to herein as ‘interactions’.

29. Surface layer: Refers herein to a chemical layer which functionally derivatizes the surface of a solid support. For instance, the surface chemical groups can be changed by the derivatization layer according to the particular chemical functionality of the derivatizing agent. In the case of solid objects used as ‘scaffolds’ for creating power nonlinear-active labels (see below), the solid surface can be derivatized to produce a different chemical functionality which can be presented to nonlinear active moieties or particles, or to targets. For instance, a silica bead with negatively charged silanol groups on its surface can be converted to an amine-reactive, amine-containing, etc. surface via organosilane reagents.

30. Delivery, Illumination, Collection: In the context of manipulation of optical radiation (e.g., light beams), delivery and illumination refer herein to the guiding of the fundamental beam to the interface or regions of interest at an interface; collection refers to the optical collection of the nonlinear light produced at the interface (e.g., second harmonic light).

31. Inhibitor, inhibiting: Defined herein as moieties, molecules, compounds or particles which bind to probes in competition with targets; the probe-target interactions are decreased or prevented in the presence of an inhibitor compound, molecule or particle. Blocking agents refers herein to those compounds, molecules, moieties or particles which prevent probe-target interactions (e.g., binding reactions between probes and targets).

32. Agonist: Defined herein as moieties, molecules, compounds or particles which activate an intracellular response when they bind to a receptor.

33. Antagonist: Defined herein as moieties, molecules, compounds or particles which competitively bind to a receptor on a cell surface at the same site as agonists, but which do not activate the intracellular response initiatied by the active form of the receptor (e.g., activated by agonist binding), and can thereby inhibit the intracellular responses of agonists or partial agonists. Antagonists do not diminish the baseline intracellular response in the absence of an agonist or partial agaonist.

34. Partial Agonist: Defined herein as moieties, molecules, compounds or particles which activate the intracellular response when they bind to a receptor on the cell surface to a lesser degree or extent than do agonists.

35. Interactions: Defined herein as some physical or chemical reaction or interaction between components in a sample. For example, the interactions can be physico-chemical binding reactions between a probe and a target, dipole-dipole attraction or repulsion between two molecules, van der Waals interactions between two atomic or molecular species, a chemical affinity interaction, a covalent bond between molecules, a non-covalent bond between molecules, an electrostatic interaction (repulsive or attractive), a hydrogen bond and others.

36. Effects: Defined herein as the measurable properties of probe-target interactions or the consequences of the interactions (e.g., secondary reactions, ion channel opening or closing, etc.). These include, the following properties, for example:

i) the intensity of the nonlinear or fundamental light.

ii) the wavelength or spectrum of the nonlinear or fundamental light.

iii) position of incidence of the fundamental light on the surface or substrate (e.g., for imaging).

iv) the time-course of either i), ii) or iii).

v) one or more combinations of i), ii), iii) and iv).

37. Time-course: Refers herein as the change in time of some measurable experimental such as light intensity or wavelength of light. Also referred to as ‘kinetics’ of some probe-target interaction, or probe-target-other component interaction for example.

38. Well-defined: In the context of ‘well-defined direction’, refers herein to the deterministic scattering of light (fundamental or nonlinear beams) from a substrate. By contrast, for example, fluorescence emission is emitted at somewhat random directions.

39. Sample: Contains the probes, targets or other molecules, particles or moieties under study by the invention. The sample contains at least one interface capable of generating the nonlinear optical light, with said interface comprised of at least one surface containing attached probes. Examples of components of samples include prisms, wells, microfluidics, substrates, buffer with targets, drugs in buffers, surfaces with attached probes. The terms ‘substrate’ and ‘surface’ are often used interchangeably herein. In some cases, the term ‘support’ can be construed to mean ‘surface’.

40. Modulator, Modulates: This term refers herein to any substance, moiety, molecule, biological component or compound which influences the kinetic or equilibrium properties of probe-target interactions (e.g., binding reaction). Modulators may change the rate of probe-target binding, the equilibrium constant of probe-target binding or, in general, enhance or reduce probe-target interactions. Examples of modulators are the following: inhibitors, drugs, small molecules, agonists and antagonists.

### GENERAL SCHEME

A general scheme for measuring probe-target interactions or their effects using the present invention is as follows:

i) Illuminate the sample with light capable of undergoing second harmonic light; in the absence of either probes or targets, or both, the intensity and/or spectrum and/or timecourse of either or both intensity or spectrum of the second harmonic light can serve as a background or baseline.

ii) Mix probes, targets, probes and targets, drugs, etc., or other components, which can modulate the probe-target interactions or their effects (at the same time or at separate times) and measure the resulting second harmonic light intensity and/or spectrum (or as a function of time, i.e., timecourse). This measured information serves as the signal for the desired interaction.

iii) A direct, optical read-out of the measured information can be performed or, optionally, the measured information can be modeled to determine, for example, kinetic or equilibrium properties of the probe-target interactions, with or without blocking agents, inhibitors, agonist, antagonist, etc.

### PREFERRED EMBODIMENT

In a preferred embodiment of the invention, the amine-reactive oxazole dye (SE) 1-(3-(succinimidyloxycarbonyl)benzyl)-4-(5-(4-methoxyphenyl)oxazol-2-yl)pyridinium bromide (PyMPO, SE: Molecular Probes Corp.) is reacted with a 1:1 molar ratio of ethylenediamine under the conditions specified by the Molecular Probes direction and is allowed to react to completion. The oxazole-based dye now contains a single amine group. This product is then reacted with the 5′-phosphate end of an oligonucleotide using the cross-linker EDAC (ethyl dimethylaminopropyl carbodiimide) according to Molecular Probes protocol resulting in a phosphoramidate bond between the dye and the oligonucleotide. Alternatively, if the oligonucleotides are chemically synthesized, an amine group can be incorporated at the 5′-end and this can be reacted directly with PyMPO, SE by following protocols published by Molecular Probes Corp.

DNA microarrays can be obtained commercially or constructed according to public literature (eg., http://cmgm.stanford.edu/pbrown/mguide/index.html). The surface chemistry to be used is that found in Chrisey, L. A. et al. (1996) in which oligonucleotides are attached to self-assembled monolayer silane films on fused silica slides. Silanization is done via N-(2-aminoethyl)-3-aminopropyltrimethoxysilane. Hybridization against labeled targets is achieved using standard protocols found in the prior art, for example as found in: Ramsay, G. DNA chips—states-of-the-art. Nature Biotechnology 1998, 16(1), 40-44; Marshall, A.; Hodgson, J. DNA chips—an array of possibilities. Nature Biotechnology 1998, 16(1), 27-31; S. A. Fodor, Science 277 (1997), 393; M. Schena et al., Science 270 (1995), 467 and references contained therein.

The DNA microarray chip is mounted on an x-y translation stage and driven by personal computer (PC control) using a motorized translator (acquired from Oriel, Inc.) or using one of the many procedures in the art (eg., V. G. Cheung et al., 1999).

Drawing 1 illustrates the nonlinear optical apparatus of the present invention. A femtosecond pulsed laser (5) (Spectra-Physics Corp.) for example, operating at 800 nm at 80 MHz with sub-100 fs pulses at >0.5 W average power is used as the source of the fundamental light [alternatively, a 10 W Argon ion laser (Coherent Corp.) can be used to pump a Ti:sapphire oscillator (Lexel Corp.) to produce the femtosecond or picosecond pulses of light]. The polarization of the fundamental can be optionally selected using a half-wave plate (10) (Melles Griot, 16 MLB 751) and focused tightly by a lens (15) on to a color filter (20) (CVI Corp., LP 780) designed to pass the fundamental light but block the nonlinear light (eg., the second harmonic). The pass filter can be an interference filter, color filter, etc. and its purpose is to prevent the second harmonic light from entering the laser cavity and causing disturbances in the lasing properties. The fundamental is then reflected from a mirror (25) and impinges at a specific location and with a specific angle on the sample surface (30). The beam diameter at the substrate surface is about 100 microns. The mirror (25) is scanned using a galvanometer-controlled mirror scanner, a rotating polygonal mirror scanner, a Bragg diffractor, acousto-optic deflector, or other means known in the art to allow control of a mirror's position. For instance, the incident angle and direction of the fundamental light on the sample surface can be varied in a known manner by the use of a precision adjustable mirror mount and a polygonal mirror using a Piezoelectric mirror mount 17 ASM 001-Melles-Griot and Mirror 02 MLQ 011/003, Melles-Griot) and driving it through the use of a stepper driven motor (17PCS001 and 17PCC001 Melles-Griot) and PC control. A galvanometer mirror or any other PC-controllable beam deflection optic can be used. For example, 16-bit galvo positioners are available from General Scanning, Inc. and Cambridge Research which use closed loop servo-control systems to achieve precise alignment control.

The silica sample surface (30) is mounted on an x-y translation stage (35) (made from stacked linear stages, Newport Corp., PM500-L and computer controlled) to select a specific location on the surface for generation of the second harmonic beam. Although FIG. 1 depicts a dry sample, the sample surface can be enclosed and in contact with liquid or buffer. The second harmonic beam intensity depends on a number of factors, including the peak electric field intensity of the fundamental which, in turn, is related to the temporal width of the pulse. Accordingly, it can be important to use ‘fast’ mirrors or lenses to minimize the dispersion of the pulse. The fundamental and second harmonic beams are scattered in well-defined directions from the silica surface. Because of this, a minimum of optics are required to collect the second harmonic light, unlike the case with fluorescence detection in which the fluorescence is emitted isotropically. The fundamental light is filtered using a color filter leaving only the second harmonic light.

The second harmonic is reflected from mirror (40) (For example: 01 MFG 033/023, Melles-Griot Corp.), sent through a pass-filter (45) (CVI Corp., BG 39) to pass the second harmonic while blocking the fundamental, and its polarization selected, if necessary, by a polarizing optic (50), then focusing the beam using a lens (55) onto a monochromator (CVI Laser Corp., CM 110) and on to a photomultiplier tube (PMT) (Hamamatsu 928P or R2658P, power supply C3830) (60). The PMT photocurrent is fed to a photon counting unit (Hamamatsu, C3866) which discriminates the signal and converts the photoelectron pulses from the PMT into 5 V digital signals which are fed, in turn, to a photon counting board for a PC (Hamamatsu, M7824) and controlled using Labview software and drivers. The beam diameter will be on the order of one element in the microarray, and the XY-location of the beam can be determined from the position of the scanning mirror and a feedback loop to the control PC; a map of the intensity of nonlinear light vs. the microarray surface location can be determined. Real-time data can also be obtained in the same manner, either at a single region within the array, or back-and-forth scanning over time between regions so as to sample the same regions over a period of time. In this manner, an intensity image and its time-dependence can be acquired for any or all regions in the microarray. Given a fixed detector and incident angle and direction and stage position, the position of the reflecting mirror can be used to determine the region of the array under illumination since the nonlinear beam will have a well-defined direction with respect to the surface. Alternatively, a CCD array detector can be used and controlling the translation stage, a mirror scanner or both and correlating their positions with the measured signal of the photodiode elements of a CCD array is disclosed in U.S. Pat. No. 6,084,991.

By translating either the stage or changing the incident position of the fundamental light, or some combination thereof, an image of second harmonic intensity from the entire array surface can be built up, assigning intensity of second harmonic light to different regions or elements within the array using a standard software program such as Labview (Labview, Inc.) or other software. The square root of the second harmonic intensity is proportional to the concentration of labelled targets which are hybridized to the probes.

### ALTERNATIVE EMBODIMENTS

In an alternative embodiment, the microarray can be in contact with, attached to, or directly patterned on, a prism capable of allowing total internal reflection at the interface containing the probes. Thus, in this mode, the fundamental beam would undergo total internal reflection at the interface containing the probes and its evanescent wave would be used to generate the nonlinear light. FIG. 2 illustrates an embodiment of this type. In FIG. 2, an index matching material or liquid (75) is used to couple the prism (70) to a substrate containing the microarray (80) in contact with solution containing targets (85), whereby total internal reflection occurs at the interface between material (80) and solution (85). The prism material can be, for example, BK7 type glass (Melles Griot) and the index matching material obtained commercially from Corning Corp. or Nye Corp.

In an alternative embodiment, the experimental set-up is as described in Salafsky and Eisenthal, 2000 and references set forth therein. A femtosecond pulsed laser (Mail-Tai, Spectra-Physics) is used as the source of fundamental light at 800 nm operating at 80 MHz with <200 fs pulses at 1 W average power. The laser beam is focused with a concave lens (Oriel) (spot size ˜1 mm²) on to the entrance aperture of a Dove prism (Melles Griot, BK-7) which is mounted in a teflon holder and in contact with solution (10 mM phosphate buffer, pH 7) or distilled water. The beam undergoes total internal reflection (evanescent wave generation) within the prism and the fundamental and second harmonic beams emerge roughly collinearly from the exit aperture. A color filter is used to block the fundamental light while passing the second harmonic to a monochromator (2 nm bandwidth slit). The monochromator is scanned from 380-500 nm to detect the second harmonic spectrum. If necessary, the fundamental light wavelength can be tuned as well. A single photon counting detector and photomultiplier tube are used to detect the output of the monochromator and a PC with software are used to record the data and control the monochromator wavelength. A background second harmonic signal is measured.

In an alternative embodiment, a planar waveguide structure 110 is used for the solid substrate (FIG. 3). In this embodiment, a thin layer of high index of refraction material 115 (the waveguide), such as TiO₂ or Ta₂O₅, is deposited on top of the substrate 110 (typically glass). A thin diffraction grating 115 is scribed into this waveguide and light from the laser 100 is coupled using this grating into the waveguide. Second harmonic light can be collected using lenses and filters and detected with either a PMT-type device or a CCD camera.

FIGS. 4a-4c illustrate an embodiment of a flow cell for carrying out probe-target reactions. The flow cell is 3220 is shown in detail. FIG. 4a is a front view, FIG. 4b is a cross sectional view, and FIG. 4c is a back view of the cavity. Referring to FIG. 4a, flow cell 3220 includes a cavity 3235 on a surface 4202 thereon. The depth of the cavity, for example, may be between about 10 and 1500 .mu.m, but other depths may be used. Typically, the surface area of the cavity is greater than the size of the probe sample, which may be about 13.times.13 mm. Inlet port 4220 and outlet port 4230 communicate with the cavity. In some embodiments, the ports may have a diameter of about 300 to 400 .mu.m and are coupled to a refrigerated circulating bath via tubes 4221 and 4231, respectively, for controlling temperature in the cavity. The refrigerated bath circulates water at a specified temperature into and through the cavity.

A plurality of slots 4208 may be formed around the cavity to thermally isolate it from the rest of the flow cell body. Because the thermal mass of the flow cell is reduced, the temperature within the cavity is more efficiently and accurately controlled.

In some embodiments, a panel 4205 having a substantially flat surface divides the cavity into two subcavities. Panel 4205, for example, may be a light absorptive glass such as an RG1000 nm long pass filter. The high absorbance of the RG1000 glass across the visible spectrum (surface emissivity of RG1000 is not detectable at any wavelengths below 700 nm) substantially suppresses any background luminescence that may be excited by the incident wavelength. The polished flat surface of the light-absorbing glass also reduces scattering of incident light, lessening the burden of filtering stray light at the incident wavelength. The glass also provides a durable medium for subdividing the cavity since it is relatively immune to corrosion in the high salt environment common in DNA hybridization experiments or other chemical reactions.

Panel 4205 may be mounted to the flow cell by a plurality of screws, clips, RTV silicone cement, or other adhesives. Referring to FIG. 4b, subcavity 4260, which contains inlet port 4220 and outlet port 4230, is sealed by panel 4205. Accordingly, water from the refrigerated bath is isolated from cavity 3235. This design provides separate cavities for conducting chemical reaction and controlling temperature. Since the cavity for controlling temperature is directly below the reaction cavity, the temperature parameter of the reaction is controlled more effectively.

Substrate 130 is mated to surface 4202 and seals cavity 3235. Preferably, the probe array on the substrate is contained in cavity 3235 when the substrate is mated to the flow cell. In some embodiments, an O-ring 4480 or other sealing material may be provided to improve mating between the substrate and flow cell. Optionally, edge 4206 of panel 4205 is beveled to allow for the use of a larger seal cross section to improve mating without increasing the volume of the cavity. In some instances, it is desirable to maintain the cavity volume as small as possible so as to control reaction parameters, such as temperature or concentration of chemicals more accurately. In additional, waste may be reduced since smaller volume requires smaller amount of material to perform the experiment.

Referring back to FIG. 4a, a groove 4211 is optionally formed on surface 4202. The groove, for example, may be about 2 mm deep and 2 mm wide. In one embodiment, groove 4211 is covered by the substrate when it is mounted on surface 4202. The groove communicates with channel 4213 and vacuum fitting 4212 which is connected to a vacuum pump. The vacuum pump creates a vacuum in the groove that causes the substrate to adhere to surface 4202. Optionally, one or more gaskets may be provided to improve the sealing between the flow cell and substrate.

FIG. 4d illustrates an alternative technique for mating the substrate to the flow cell. When mounted to the flow cell, a panel 4290 exerts a force that is sufficient to immobilize substrate 130 located therebetween. Panel 4290, for example, may be mounted by a plurality of screws 4291, clips, clamps, pins, or other mounting devices. In some embodiments, panel 4290 includes an opening 4295 for exposing the sample to the incident light. Opening 4295 may optionally be covered with a glass or other substantially transparent or translucent materials. Alternatively, panel 4290 may be composed of a substantially transparent or translucent material.

In reference to FIG. 4a, panel 4205 includes ports 4270 and 4280 that communicate with subcavity 3235. A tube 4271 is connected to port 4270 and a tube 4281 is connected to port 4280. Tubes 4271 and 4281 are inserted through tubes 4221 and 4231, respectively, by connectors 4222. Connectors 4222, for example, may be T-connectors, each having a seal 4225 located at opening 4223. Seal 4225 prevents the water from the refrigerated bath from leaking out through the connector. It will be understood that other configurations, such as providing additional ports similar to ports 4220 and 4230, may be employed.

Tubes 4271 and 4281 allow selected fluids to be introduced into or circulated through the cavity. In some embodiments, tubes 4271 and 4281 may be connected to a pump for circulating fluids through the cavity. In one embodiment, tubes 4271 and 4281 are connected to an agitation system that agitates and circulates fluids through the cavity.

Referring to FIG. 4c, a groove 4215 is optionally formed on the surface 4203 of the flow cell. The dimensions of groove, for example, may be about 2 mm deep and 2 mm wide. According to one embodiment, surface 4203 is mated to the translation stage. Groove 4211 is covered by the translation stage when the flow cell is mated thereto. Groove 4215 communicates with channel 4217 and vacuum fitting 4216 which is connected to a vacuum pump. The pump creates a vacuum in groove 4215 and causes the surface 4203 to adhere to the translation stage. Optionally, additional grooves may be formed to increase the mating force. Alternatively, the flow cell may be mounted on the translation stage by screws, clips, pins, various types of adhesives, or other fastening techniques.

In a further alternative embodiment, a suspension of beads, cells, liposomes or other objects are the probes (130), or comprise probes attached thereto, as shown in FIG. 5. The scattered nonlinear light from such a sample—eg., an isotropic sample in which each individual beads or other objects are about a coherence length or farther apart—is generated in all directions with some distribution in intensity. Fundamental light is transmitted through the suspension (130) and the nonlinear radiation collected. A number of modes of collecting the scattered nonlinear light are available. For example, collection of the second harmonic can be in the forward direction (A), at a right angle to the fundamental light (B), or using an integrating sphere approach (C). Part C shows an integrating sphere 165 with the sample 150 placed inside. Fundamental light (145) enters the entrance port (170), passes through the sample (150), undergoes a reflection at the sphere wall, and is stopped by baffle (175). The scattered second harmonic light is collected from the sphere surface through exit port (155) and coupled out of the sphere by a fiber optic line (160). Beads can support phospholipid bilayers (eg., with membrane proteins) or probes such as proteins or nucleic acids can be attached to their surface. The beads provide a large amount of distributed surface area in the sample and can be a useful alternative to planar surface geometries, especially when the fundamental and nonlinear light is used in the transmission mode.

In an alternative embodiment (FIG. 6), the excitation light is tranformed from a point-like shape into some other shape using various optics. For instance, the point-like beam shape of the fundamental beam can be transformed into a line shape, useful for scanning the sample surface. However, because the intensity of the nonlinear beam depends on, among other factors, the intensity of the fundamental (typically a quadratic dependence on the fundamental intensity), this transformation will result in less nonlinear light intensity generated at a given location. To generate a line-shape in the fundamental (which can typically be a round point of ˜2 mm diameter), one can direct the fundamental beam into a microscope objective which has a magnification power of about 10 followed by a 150 mm achromat to collimate the beam as well known in the prior art and as disclosed in detail in U.S. Pat. No. 5,834,758. As shown in FIG. 6, the fundamental light 180 is a beam of typically 2-3 mm diameter. This beam is directed through a microscope objective 185. The objective, which has a magnification power of 10, expands the beam to about 30 mm. The beam then passes through a lens 190. The lens, which can be a 150 mm achromat, collimates the beam. Typically, the radial intensity of the expanded collimated beam has a Gaussian profile. To minimize intensity variations in the beam, a mask 195 can be inserted after lens 190 to mask the top and bottom of the beam, thereby passing only the central portion of the beam. In one embodiment, the mask passes a horizontal band that is about 7.5 mm. Thereafter, the beam passes through a cylindrical lens 200 having a horizontal cylinder axis, which can be a 100 mm f.1. made by Melles Griot. The cylindrical lens expands the beam spot vertically. Alternatively, a hyperbolic lens can be used to expand the beam vertically while resulting in a flattened radial intensity distribution. From the cylindrical lens, the light passes through a lens 205. Optionally, a planar mirror can be inserted after the cylindrical lens to reflect the excitation light toward lens 205. To achieve a beam height of about 15 mm, the ratio of the focal lengths of the cylindrical lens 200 and lens 205 is approximately 1:2, thus magnifying the beam to about 15 mm. Lens 205, which in some embodiments is a 80 mm achromat, focuses the light to a line of about 15 mm.×50 microns at the sample surface 210.

In an alternative embodiment shown in FIG. 7, probes patterned in a two-dimensional array (A, top view of array on surface) where each region on the surface—{1,35} in this example—can be a different oligonucleotide or protein sequence (or a combination of the same and different sequences) and labeled targets are used to detect binding. Part B shows a side-view of the sample surface (220) in a well (215) containing the targets (225) shown here as protein objects with second-harmonic-active labels (X) attached. The well can hold liquid or buffer and serves to physically separate the contents of the well from other parts of the substrate or other elements in a substrate array. The fundamental light can be multiplexed and each resultant beam can be guided by individual mirrors to simultaneously scan different lines or regions within the array, thus increasing even further the potential of the technique for high-throughput studies.

In an alternative embodiment, the method of Levicky et al. or the method of L. A. Chrisey et al. is used to attach the probe DNA to the substrate. In the method of Chrisey as illusrated in FIG. 8, a fused silica or oxidized silicon substrate is used (230) and derivatized with N-(2-aminoethyl)-3-aminopropyltrimethoxysilane (EDA) (235). In one embodiment, the EDA-modified surface is then treated with the heterobifunctional crosslinker (SMPB), whose succinimide ester moiety reacts with the primary amino group of EDA (240). A thiol-DNA oligomer subsequently (245) of base-pair sequence (xzzy) (where ‘xzzy’ represents the entire sequence) reacts with the maleimide portion of the SMPB crosslinker, to yield the covalently bound species shown (250).

In an alternative embodiment, elements in the surface array are physically separated as illustrated in FIG. 9, allowing for different targets, target solutions, etc. to be added selectively to any or all of the elements. Part (A) is a top-view of the substrate (255) with partitions or walls (260) separating the different well regions—in this example, 16 wells. Part (B) shows a side-view of a well (265) with attached probes (270). Such arrays are commonly found in the art, such as the 96-well plates, etc. and are commercially available (Fisher Scientific, Inc. etc.)

In an alternative embodiment, a glass substrate surface can be coated with a layer of a reflective metal such as silver. The metallic layer will increase nonlinear optical generation and collection. Biomolecules or other particles can be attached to derivatized layers built on top of the metal. For instance, the metal can be coated with a layer of silicon dioxide (SiO₂), then with a layer of aminosilane such as 3-amino-octyl-trimethoxysilane. Oligonucleotides or polynucleotides can then be attached to the aminosilane layer using linkers which connect the 3′ or 5′ end of the oligo to the amine group. Alternatively, the oligos or polynucleotides can be adsorbed to the aminosilane layer. FIG. 10 illustrates an embodiment of this type where a glass substrate (275) is derivatized with a Ag layer (280). A thin coat of SiO₂ is then deposited on top of the silver layer (285) and derivatized with the aminosilane (290).

In an alternative embodiment, bead-based fiber-optic arrays can be used (ref. 34) in which light beams (eg., fundamental and second harmonic) travel via total internal reflection along the path of the fiber. The fundamental light is coupled into the bundle or individual optical fibers and second harmonic light is generated at the tip surface and collected back through the fiber. In this embodiment, individual optical fibers can be converted into DNA sensors by attaching a DNA probe to the distal tip (ref. 17,18) or by removing the cladding of the optical fiber and attaching the DNA probe to the outside of the core (ref. 19-22). Simple DNA arrays can be made from such optical fibers by physically bundling multiple fibers together (ref. 23). There are many variations on this theme, for example by selectively etching the distal-end cladding to create wells of different depths at the distal end of the fiber, where the tip of the fiber constitutes the bottom of the wells (ref. 24). Latex or silica beads can then be loaded into the wells (ref. 25). Fiber-optic oligonucleotide arrays can be prepared by attaching DNA probes to microspheres and then filling each well with a microsphere carrying a different DNA probe. Each different type of microsphere is tagged with a unique combination of fluorescent dyes or DNA probes either before or after probe attachment (refs. 26,27). ‘Zip codes’ for universal fabrication (ref. 29) and molecular beacons (refs. 28,30) for label-less detection can also be used with the optical sensor-beaded arrays. FIG. 11 illustrates a fiber-optic bundle array. Part (A) shows a bundle of fiber optic cables (295) with wells at the distals ends for placement of beads (300). Part (B) shows a close-up view of a single optical fiber. Fundamental light travels (ω) toward the distal end with the bead (305). Some fundamental light is scattered back from the bead along with second harmonic light (2ω) and travels back through the fiber to the proximal end where an optical train and detection system (not shown) separates the fundamental radiation from the second harmonic radiation. Bead (310) is covered with attached probes.

In an alternative embodiment, the detector (65) of the nonlinear radiation in FIG. 1 is a photomultiplier tube operated in single-photon counting mode. Photocurrent pulses can be voltage converted, amplified, subjected to discrimination using a Model SR445 Fast Preamplifier and Model SR 400 Discriminator (supplied by Stanford Research Systems, Inc.) and then sent to a counter (Model 3615 Hex Scaler supplied by Kinetic Systems). Photon counter gating and galvo control through a DAC output (Model 3112, 12-Bit DAC supplied by Kinetic Systems) can be synchronized using a digital delay/pulse generator (Model DG535 supplied by Stanford Research Systems, Inc.). Communication with a PC computer 29 can be accomplished using a parallel register (Model PR-604 supplied by DSP Technologies, Inc.), a CAMAC controller card (Model 6002, supplied by DSP Technologies, Inc.) and a PC adapter card (Model PC-004 supplied by DSP Technologies, Inc.).

In an alternative embodiment, a bandpass, notch, or color filter is placed in either or all of the beam paths (eg., fundamental, second harmonic, etc.) allowing, for example, for a wider spectral bandwidth or more light throughput.

In an alternative embodiment, an interference, notch-pass, bandpass, reflecting, or absorbant filter can be used in place of the filters in the figures in order to either pass or block the fundamental or nonlinear optical beams.

According to another embodiment, detection of the nonlinear optical light is achieved using a charge coupled detector (CCD) in place of a photomultiplier tube or other photodetector. The CCD subsystem communicates with and is controlled by a data acquisition board installed in a computer. Data acquisition board may be of the type that is well known in the art such as a CIO-DAS 16/Jr manufactured by Computer Boards Inc. The data acquisition board and CCD subsystem, for example, may operate in the following manner. The data acquisition board controls the CCD integration period by sending a clock signal to the CCD subsystem. In one embodiment, the CCD subsystem sets the CCD integration period at 4096 clock periods. by changing the clock rate, the actual time in which the CCD integrates data can be manipulated. During an integration period, each photodiode accumulates a charge proportional to the amount of light that reaches it. Upon termination of the integration period, the charges are transferred to the CCD's shift registers and a new integration period commences. The shift registers store the charges as voltages which represent the light pattern incident on the CCD array. The voltages are then transmitted at the clock rate to the data acquisition board, where they are digitized and stored in the computer's memory. In this manner, a strip of the sample is imaged during each integration period. Thereafter, a subsequent row is integrated until the sample is completely scanned.

In an alternative embodiment, one is interested in finding drugs, antagonists, agonists or other species which block or reduce the binding of probes with targets—these compounds may be referred to as ‘inhibitors’. In this application, labeled targets are bound to probes at the interface. The inhibitors are added to the sample, and if the particular species being tested is successful in blocking or reducing the probe-target binding, the nonlinear optical light measured will change—the background radiation in this embodiment is due to target-probe binding; the displacement of the targets from the probes at the interface by the inhibitors leads to a change in the nonlinear optical light measured, for instance as a decrease in intensity of the nonlinear radiation generated by the interface or a wavelength shift in the nonlinear radiation spectrum.

In an alternative embodiment, the nonlinear spectrum of a sample is measured by measuring the nonlinear radiation (e.g., second harmonic radiation) at two or more spectral points or bands, using a monochromator, filter or other wavelength-selecting device to accomplish this.

In an alternative embodiment, a monochromator (60) can be placed before the detecting element in the device, in order to spectrally resolve the nonlinear optical radiation (FIG. 1).

In an alternative embodiment, nucleic acid or PNA microarrays can be obtained commercially or constructed according to public literature (eg., http://cmgm.stanford.edu/pbrown/mguide/index.html). The surface chemistry to be used is that found in Chrisey, L. A. et al. (1996) in which oligonucleotides are attached to self-assembled monolayer silane films on fused silica slides. Silanization is accomplished via N-(2-aminoethyl)-3-aminopropyltrimethoxysilane.

In other embodiments, oligonucleotides or PNAs can be attached to the solid substrate via light-directed synthesis (Fodor et al., 1997) or via chemical synthesis (e.g., Chrisey, L. A., 1996).

In stilll other embodiments, surfaces or microarrays microarrays of oligonucleotides or PNAs can be obtained commercially or constructed according to public literature (eg., http://cmgm.stanford.edu/pbrown/mguide/index.html).

DNA microarrays can be obtained commercially or constructed according to public literature (eg., http://cmgm.stanford.edu/pbrown/mguide/index.html). The surface chemistry to be used is that found in Chrisey, L. A. et al. (1996) in which oligonucleotides are attached to self-assembled monolayer silane films on fused silica slides. Silanization is done via N-(2-aminoethyl)-3-aminopropyltrimethoxysilane.and Hoheisel, J. D. “Improved solid supports and spacer/linker systems for the synthesis of spatially addressable PNA-libraries” Nucleosides Nucleotides 18 (1999) 1289-1291 on glass or silica. The buffer or solution in contact with the PNA oligonucleotides can be chosen from a range of those known in the art. Hybridization and wash solutions are found in the art. For example, the web site: cmgm.stanford.edu/pbrown/protocols gives detailed instructions for probe-target hybridization.

Microarrays can be mounted on an x-y translation stage and driven by personal computer (PC control) using a motorized translator (acquired from Oriel, Inc.) or using one of the many procedures in the art (eg., V. G. Cheung et al., 1999).

In an alternative embodiment, imaging techniques described in the art (Peleg, 1999 or Campagnola et al.) can be performed using SHG-labeled components (such as labeled ligands or receptors) instead of the membrane-intercalating dyes used the art. These imaging techniques iclecan be used to image solid surfaces, cell surfaces or other interface using SHG-labeled components.

In an alternative embodiment, the nonlinear optical measurements can be made in the presence of labelled targets in solution, liquid or buffer in contact with the substrate with attached probes (e.g., no washing step is required to remove non-bound labelled targets).

In an alternative embodiment, channels (or microfluid) channels can be used to introduce the components into the sample cell via positive displacement, pumping, electrophoretic means or other means known in the art for manipulating the flow of components into and out of a reaction chamber.

In an alternative embodiment, the kinetics of some probe-target binding reaction are to be measured at some concentration of target. In this embodiment, the timecourse of the intensity and/or spectrum of the nonlinear optical light are measured. The measured information can be converted into a timecourse of bound target concentration (e.g., probe-target concentration in mM/s or μM/s). Drugs or other enhancers or reducers, for example, of the probe-target binding equilibrium or kinetic rate of formation can be used so as to compare the effect of the added substance on the probe-target reactions.

In an alternative embodiment, the apparatus can be assembled into a user-closed product with a user-controlled interface (an LED panel, for example, or PC-based software) with the option of inserting and removing disposable substrates (e.g., biochips) with the attached probes.

In an alternative embodiment, the labels can be photoactivated or photomodulated with a beam of light (e.g., not the fundamental) such that, upon irradiation of the sample with the beam of light, the labels become nonlinear optical active (or more or less nonlinear optical active). The beam of light can, for example, cleave a chemical bond (e.g., using UV light), well known in the art as ‘caged’ compounds.

In an alternative embodiment, a photodiode, avalanche photodiode or other photoelectric detector (65) in FIG. 1 is used as the light detection means.

In an alternative embodiment, the surface array can be in a fixed position and the incident light beam scanned across the surface using methods well known in the art, such as a galvanometer mirror or a polygonal mirror.

In an alternative embodiment, the scanning method can be a combination of both stage translation (x-y) and beam scanning, wherein the latter controls the incident position of the fundamental beam on the array surface.

In an alternative embodiment, a stop-flow mixing chamber is used to rapidly mix the components in the sample cell.

In an alternative embodiment, probe-target interactions with labelled targets can be imaged on some surface such as a tissue surface, patterned cells on a surface, surface-attached probes (e.g., microarrays or arrays of DNA, protein, etc.); the imaging can occur in-vitro or in-vivo. In cases of in-vivo imaging, the imaging can be performed using endoscopes or other instruments known in the art for introducing and collect light in-vivo.

In an alternative embodiment, a biological probe-target binding reaction can be measured in the presence of agonists, antagonists, drugs, or small molecules which can modulate the binding strength (e.g., equilibrium constant) of the said probe-target binding reaction. This embodiment can be useful in many cases, for example when one would like to know the efficacy of a drug's ability to block a certain probe-target reaction for medical uses or basic research.

In an alternative embodiment, the proportionality constant (calibration curve of intensity of second harmonic light vs. concentration of targets bound to attached probes) is determined by measuring the concentration of targets using another method such as radiolabeling or fluorescence labels of the targets. Once the calibration curve is known, for a given probe and target type (e.g., cDNA, RNA, size of oligos, etc.), the concentration of bound target is determined using this relation and the measured second harmonic intensity.

In an alternative embodiment, the nonlinear optical, surface-selective apparatus can comprise a unit without the light excitation source (e.g., with sample compartment, filters, detectors, monochromator, computer interface, software, or other parts) so that the user can supply his own excitation source and adapt its use to the methods described herein.

In an alternative embodiment, the measurable information can be recorded in real time.

In an alternative embodiment, target-probe interactions can be measured in the presence of some modulator of the interactions—the modulator being, for example, a small molecule, drug, or other moiety, molecule or particle which changes in some way the target-probe interactions (e.g., blocks, inhibits, etc.). The modulator can be added before, during or after the time in which the probe-target interactions occur.

Various Configurations of an Apparatus Using the Surface-selective Nonlinear Optical Technique for Detection of Probe-target Reactions.

The apparatus for detection of the probe-target reactions or their effects can assume a variety of configurations. In its most simple form, the apparatus will comprise the following:

i) a source of the fundamental light

ii) a substrate or sample with surface-attached probes

iii) a detector for measuring the intensity of the second harmonic or other nonlinear optical beams.

More elaborate versions of the apparatus will employ, for example: a monochromator (for wavelength selection), a pass-filter, color filter, interference or other spectral filter (for wavelength selection or to separate the fundamental(s) from the higher harmonics), one or more polarizing optics, one or more mirrors or lenses for directing and focusing the beams, computer control, software, etc.

The mode of delivering or generating the nonlinear optical light (e.g., SHG) can be based on one or more of the following means: TIR (Total internal reflection), Fiber optics (with or without attached beads), Transmission (fundamental passes through the sample), Reflection (fundamental is reflected from the sample), scanning imaging (allows one to scan a sample), confocal imaging or scanning, resonance cavity for power build-up, multiple-pass set-up.

Measured information can take the form of a vector which can include one or more of the following parameters: {intensity of light (typically converted to a photovoltage by a PMT or photodiode), wavelength of light (determined with a monochromator and/or filters), time, substrate position (for array samples, for instance, where different sub-samples are encoded as function of substrate location and the fundamental is directed to various (x,y) locations}. Two general configurations of the apparatus are: image scanning (imaging of a substrate—intensity, wavelength, etc. as a function of x,y coordinate) and spectroscopic (measurement of the intensity, wavelength, etc. for some planar surface or for a suspension of cells, liposomes or other particles).

The fundamental beam can be delivered to the sample in a variety of ways. FIGS. 12-16 are schematics of various modes of delivering the fundamental and generating second harmonic beams. It is understood that in sum- or difference-frequency configurations, the fundamental beams will be comprised of two or more beams, and will generate, at the interfaces, the difference or sum frequency beams. For the purposes of illustration, only the second harmonic generation case is described in detail herein. Furthermore, it shall be understood that the sample cell 3 in all cases can be mounted on a translation stage (1-, 2-, or 3-dimensional degrees of freedom) for selecting precise locations of the interfacial interaction volume. The sample cell in all cases can be fitted with flow ports and tubes which can serve to introduce (or flush out) components such as molecules, particles, cells, etc.

Transmission

FIG. 12A is a schematic of a configuration relying on transmission of the fundamental and second harmonic beams. The fundamental 320 (ω) passes through the sample cell 330 and interacts within a volume element (denoted by the circle) in which are contained one or more interfaces capable of generating the second harmonic beam 325 (2ω). The fundamental and second harmonic beams are substantially co-linear as denoted by beam 325. The sample cell can contain suspended beads, particles, liposomes, biological cells, etc. in some medium, providing interfacial area capable of generating second harmonics in response to the fundamental beam. As shown, the second harmonic is detected co-linearly with the fundamental direction, but could alternatively be detected off-angle from the fundamental, for instance at 90° to the fundamental beam.

FIG. 12B is a schematic of another configuration relying on transmission of the fundamental and second harmonic beams. The fundamental 335 is directed onto a sample cell 345 and the second harmonic waves are generated at the top surface—this surface can be derivatized with immobilized probes or with adsorbed particles, liposomes, cells, etc. The second harmonic waves 340 are generated within a volume element denoted by the circle at the interface between the top surface and the medium contained within cell.

FIG. 12C is a schematic of a configuration substantially similar to the one depicted in FIG. 2A except that the bottom surface of the sample cell 3, rather than the top, is used to generate the second harmonic waves.

Total Internal Reflection

FIG. 13A is a schematic of a waveguide 4 capable of acting as a total internal reflection waveguide which refracts the fundamental 365 and directs it to a location at the interface between the waveguide 380 and a sample cell 375. At this location, denoted by the circle, the fundamental will generate the second harmonic waves and undergo total internal reflection; the second harmonic beam will propagate substantially colinearly with the fundamental and exit the prism 380. Waveguide 380 will typically be in contact with air. In this illustration, the waveguide 380 is a Dove prism.

FIG. 13B is a schematic of a configuration similar to the one depicted in FIG. 13A except that the waveguide 400 allows for multiple points of total internal reflection between the waveguide 4 and the sample cell 395, increasing the amount of second harmonic light generated from the fundamental beam.

Fiber Optic

FIG. 14 depicts various configurations of a fiber optic means of delivering or collecting the fundamental or second harmonic beams. In FIG. 14A, the coupling element 410 between a source of the fundamental wave and the fiber optic is depicted. The fundamental, thus coupled into the fiber optic waveguide 405, proceeds to a sample cell 415. In FIG. 14A, the tip of the fiber can serve as the interface of interest capable of generating second harmonic waves, or the tip can serve merely to introduce the fundamental beam to the sample cell containing suspended cells, particles, etc. In FIG. 14A, the second harmonic light is collected back through the fiber optic.

FIG. 14B is identical to FIG. 14A except that a bead is attached to the tip of the fiber optic (according to means well known in the art). The bead can serve to both improve collection efficiency of the second harmonic light or be derivatized with probes or adsorbed species and presenting an interface with the medium of sample cell 425 capable of generating the second harmonic light.

FIG. 14C is identical to both FIGS. 14A and 14B except that collection of the second harmonic light is effected using a solid-angle detector 450.

Optical Resonance Cavity

An optical resonance cavity is defined between at least two reflective elements and has an intracavity light beam along an intracavity beam path. The optical cavity or resonator consists of two or more mirrored surfaces arranged so that the incident light can be trapped bouncing back and forth between the mirrors. In this way, the light inside the cavity can be many orders of magnitude more intense than the incident light. This phenomenon is well known and has been exploited in various ways (see, for example, Yariv A. “Introduction to Optical Electronics”, 2^{nd} Ed., Holt, Reinhart and Winston, N.Y. 1976, Chapter 8). The sample cell can be present in the optical cavity or it can be outside the optical resonance cavity.

FIG. 15 is a schematic of an optical resonance power build-up cavity configuration. FIG. 15A is a schematic of an optical resonance cavity in which the sample cell 465 is positioned intracavity and the fundamental and second harmonic beams are transmitted through it—a useful configuration for sample cells containing suspended particles, cells, beads, etc. The fundamental beam 455 enters the optical resonance cavity at reflective optic 460 and builds up in power between reflective elements 460 and 462 (intracavity beam). Mirror 460 is preferably tilted (not perpendicular to the direction of the incident fundamental 455) to prevent direct reflection of the intracavity beam back into the light source. The natural reflectivity and transmisivity of 460 and 462 can be adjusted so that the fundamental builds up to a convenient level of power within the cavity. The fundamental generates second harmonic light in a volume element within the sample cell denoted by the circle. Reflective optic 460 can reflect the fundamental and the second harmonic, while reflective optic 462 will substantially reflect the fundamental but allow the pass-through of the second harmonic beam 475 which is subsequently detected. U.S. Pat. No. 5,432,610 (King et al.) describes a diode-pumped power build-up cavity for chemical sensing and it and the references it makes are hereby incorporated by reference herein.

FIG. 15B is a schematic of an optical resonance power build-up cavity configuration in which the fundamental beam 475 enters the optical cavity by reflection from optic 480. A second reflective optic element 482 defines the optical resonance cavity. Element 490 is a waveguide (such as a prism) in contact with the sample cell 485 and allows total internal reflection of the fundamental beam at the interface between the waveguide and sample cell surfaces, generating the second harmonic light. Element 482 substantially reflects the fundamental beam but passes through the second harmonic beam 495 which is subsequently detected.

Reflection

FIG. 16A is a schematic of a configuration involving reflection of the fundamental and second harmonic beams. A substrate 525 is coated with a thin layer of a reflective material 520, such as a metal, and on top of this is deposited at layer 515 suitable for attachment of the probes or adsorption of particles, cells, etc. (e.g., SiO₂). This layer is in contact with the sample cell 510. The fundamental 500 passes through the sample cell 510 and generates a second harmonic wave at the interface between layers 515 and 520. The fundamental and second harmonic waves 505 are reflected back from the surface of layer 520.

FIG. 16B is substantially similar to FIG. 15A except that the second harmonic and fundamental beams are reflected 535 from the interface between the medium contained in sample cell 540 and layer 545. Layer 545 is reflective or partly reflective layer deposited on substrate 550 and is suitable for adsorption of particles, cells, etc. or attachment of probes.

FIG. 16C is a schematic illustrating that only the sample cell 565 need be used for a reflective geometry. The sample cell 565 is partly filled with some medium 570 and the fundamental and second harmonic beams are reflected 560 from the gas-liquid or vapor-liquid interface at the surface of 570.

Modes of Detection

Charge-coupled detectors (CCD) array detectors can be particularly useful when information is desired as a function of substrate location (x,y). CCDs comprise an array of pixels (i.e., photodiodes), each pixel of which can independently measuring light impinging on it. For a given apparatus geometry, nonlinear light arising from a particular substrate location (x,y) can be determined by measuring the intensity of nonlinear light impinging on a CCD array location (Q,R) some distance from the substrate—this can be determined because of the coherent, collimated (and generally co-propagating with the fundamental) nonlinear optical beam) compared with the spontaneous, stochastic and multidirectional nature of fluorescence emission. With a CCD array, one or more array elements {Q,R} in the detector will map to specific regions of a substrate surface, allowing for easy determination of information as a function of substrate location (x,y). Photodiode detector and photomultiplier tubes (PMTs), avalanche photodiodes, phototransistors, vacuum photodiodes or other detectors known in the art for converting incident light to an electrical signal (i.e., current, voltage, etc.) can also be used to detect light intensities. For CCD detector, the CCD communicates with and is controlled by a data acquisition board installed in the apparatus computer. The data acquisition board can be of the type that is well known in the art such as a CIO-DAS16/Jr manufactured by Computer Boards Inc. The data acquisition board and CCD subsystem, for example, can operate in the following manner. The data acquisition board controls the CCD integration period by sending a clock signal to the CCD subsystem. In one embodiment, the CCD subsystem sets the CCD intregration period at 4096 clock periods. By changing the clock rate, the actual time in which the CCD integrates data can be manipulated. During an integration period, each photodiode accumulates a charge proportional to the amount of light that reaches it. Upon termination of the integration period, the charge is transferred to the CCD's shift registers and a new integration period commences. The shift registers store the charges as voltages which represent the light pattern incident on the CCD array. The voltages are then trasmitted at the clock rate to the data acquisition board, where they are digitized and stored in the computer's memory. In this manner, a strip of the sample is imaged during each integration period. Thereafter, a subsequent row is integrated until the sample is completely scanned.

Sample Substrates and Sample Cells

Sample substrates and cells can take a variety of forms drawing from, but not limited to, one or more of the following characteristics: fully sealed, sealed or unsealed and connected to flow cells and pumps, integrated substrates with a total internal reflection prism allowing for evanescent generation of the nonlinear beam, integrated substrates with a resonant cavity for fundamental power build-up, an optical set-up allowing for multiple passes of the fundamental for increased nonlinear response, sample cells containing suspended biological cells, particles, beads, etc.

Data Analysis

Data analysis operates on the vectors of information measured by the detector. The information can be time-dependent and kinetic. It can be dependent on the concentration of one or more biological components, inhibitors, antagonists, agonists, drugs, small molecules, etc. which can be changed during a measurement or between measurements. It can also be dependent on wavelength, etc. In general, the intensity of nonlinear light will be transformed into a concentration or amount of a particular state (for example, the surface-associated concentration of a component or the amount of opened or closed ion-channels in cell membranes). In one example, the production of second harmonic light follows the equation:

(I_{SH})^{0.5}∝E_{2ω}=A_{χ}⁽²⁾+BΦ_{0χ}⁽³⁾ (1)

where I_{SH} is the intensity of the second harmonic light, E_{2ω} is the electric-field amplitude of the second harmonic light, A and B are constants specific to a given interface and sample geometry, Φ₀ is the electric surface potential, and _{χ}⁽²⁾ and _{χ}⁽³⁾ are the second and third-order nonlinear susceptibility tensors. _{χ}⁽²⁾ is proportional to N (surface-bound or probe-bound targets) and the hyperpolarizability per target. Surface binding reactions can follow a Langmuir-type equation:

dN/dt=k₁(C−N)/55.5*(Nₘₐₓ−N)−k₋₁N (2)

with N the amount of the targets binding to the surface (e.g., targets binding to probes), Nₘₐₓ the maximum number of the binding species at the surface at equilibrium, k₁ the association rate constant, k₁ the dissociation rate constant, dN/dt the instantaneous rate of change of the amount of surface-bound targets and C the bulk concentration of the species. Modified Langmuir equations or other equations used in determining the amount of surface-bound species in the art can also be used in the data analysis.

The details of the data analysis will depend on each specific case. If the polarization response due to a net charge on the surface —_{χ}⁽³⁾— is present, it can be subtracted out in making the measurement. Thus, the number of surface-bound species N can be directly calculated from the second harmonic intensity in this manner. Kinetics or equilibrium properties can be determined from N (at equilibrium or in real time) according, for example, to equation 2 and procedures well known in the art. There are a number of relevant papers in the art which describe this process including, for example: J. S. Salafsky, K. B. Eisenthal, “Second Harmonic Spectroscopy: Detection and Orientation of Molecules at a Biomembrane Interface”, Chemical Physics Letters 2000, 319, 435 and Eisenthal, K. B. “Photochemistry and Photophysics of Liquid Interfaces by Second Harmonic Spectroscopy” J. Phys. Chem. 1996, 100, 12997.

For probe-target processes which result directly or indirectly in changes in surface charge density or potential (an example of the indirect type is in ion-channel experiments with ion channels in a cell, where a target binds to a probe, leading to the modulation of an ion channel's dynamics which leads, in turn, to the surface charge density). In this case, labels attached to the surface of a cell are used to sense the ion channel's dynamics or state via the effect the surface charge density has on the nonlinear properties of the labels.

Data Analysis

Data analysis operates on the vectors of information measured by the detector. The information can be time-dependent and kinetic. It can be dependent on the concentration of one or more biological components (probes, targets, drugs, etc.), which can be changed during a measurement or between measurements. It can also be dependent on wavelength, etc. In general, the intensity of nonlinear light will be transformed into a concentration or amount of a particular state. In one example, the production of second harmonic light follows the equation:

(I_{SH})^{0.5}∝E_{2ω}=A_{χ}⁽²⁾+BΦ_{0χ}⁽³⁾ (1)

where I_{SH} is the intensity of the second harmonic light, E_{2ω} is the electric-field amplitude of the second harmonic light, A and B are constants specific to a given interface and sample geometry, Φ₀ is the electric surface potential, and _{χ}⁽²⁾ and _{χ}⁽³⁾ are the second and third-order nonlinear susceptibility tensors. Surface binding reactions can follow a Langmuir-type equation:

dN/dt=k₁(C−N)/55.5*(Nₘₐₓ−N)−k₋₁N (2)

with N the amount of the targets binding to the surface (e.g., targets binding to probes) and dN/dt the instantaneous rate of targets binding to the surface, Nₘₐₓ the maximum number of the binding species at the surface at equilibrium, k₁ the association rate constant, k₁ the dissociation rate constant, dN/dt the instantaneous rate of change of the amount of surface-bound targets and C the bulk concentration of the species. Modified Langmuir equations or other equations used in determining the amount of surface-adsorbed or surface-bound species in the art can also be used in the data analysis.

The details of the data analysis will depend on the specific details of each case. The number of labeled, surface-bound species N can be directly calculated from the second harmonic intensity. Kinetics or equilibrium properties can be determined from N (at equilibrium or in real time) according, for example, to equation 2 and procedures well known in the art. There are a number of relevant papers in the art which describe this process in detail including, for example: J. S. Salafsky, K. B. Eisenthal, “Second Harmonic Spectroscopy: Detection and Orientation of Molecules at a Biomembrane Interface”, Chemical Physics Letters 2000, 319, 435 and Eisenthal, K. B. “Photochemistry and Photophysics of Liquid Interfaces by Second Harmonic Spectroscopy” J. Phys. Chem. 1996, 100, 12997.

### BRIEF DESCRIPTION OF THE DRAWINGS

FIG. 1 depicts one embodiment of the apparatus in which the mode of generation and collection of the second harmonic light is by reflection off the substrate with surface-attached probes.

FIG. 2 depicts one embodiment of an apparatus in which the mode of generation and collection of the second harmonic light is by total internal reflection through a prism. The prism is coupled by an index-matching material to a substrate with surface-attached probes.

FIG. 3 depicts one embodiment of an apparatus in which the mode of generation and collection of the second harmonic light is by total internal reflection through a wave-guide with multiple reflections as denoted by the dashed line inside the wave-guide.

FIG. 4 depicts one embodiment of a flow-cell for delivery and removal of biological components and other fluids to the substrate containing attached probes.

FIG. 5 depicts three embodiments of an apparatus in which the mode of generation and collection of the second harmonic light is by transmission through a sample. In FIG. 5A, the second harmonic beam is co-linear with the fundamental. In FIG. 5B, the second harmonic is collected from a direction orthogonal to the fundamental (‘right-angle collection’). In FIG. 5C, the second harmonic light is collected by an integrating sphere and a fiber optic line.

FIG. 6 depicts an embodiment of the transformation, using a series of optical components, of a collimated beam of the fundamental light into a line shape suitable for scanning a substrate.

FIG. 7A depicts an embodiment of a substrate surface (containing attached probes) which has been patterned into an array format (elements 1-35). FIG. 7B depicts one element of a substrate array in which each element is a well with walls, with surface-attached probes, and the well is capable of holding some liquid and serves to physically separate the well's contents from adjacent wells or other parts of the substrate.

FIG. 8 depicts one embodiment of a surface chemistry used to attach oligonucleotide or polynucleotide samples to the substrate surface.

FIG. 9A depicts an embodiment of a substrate containing multiple wells (1-16), each of which contains surface-attached probes as depicted in FIG. 9B.

FIG. 10 depicts an embodiment of the apparatus substrate with the use of an aminosilane surface-attached layer on top of a reflective coating. The reflective coating underneath the aminosilane layer improves collection of the nonlinear optical light. The aminosilane layer is suitable for coupling biomolecules or other probe components to the substrate.

FIG. 11 depicts an embodiment of an apparatus in which the mode of generation and collection of the second harmonic light is through a fiber optic. FIG. 11A depicts the use of a bundle of fiber optic lines and FIG. 11B depicts the use of beads coupled to the end of a fiber for attaching probes.

FIG. 12 depicts three embodiments of an apparatus in which the mode of generation and collection of the second harmonic light is by transmission through a sample. FIG. 12A depicts both the fundamental and second harmonic beams travelling co-linearly through a sample. FIG. 12B depicts the fundamental and second harmonic beams being refracted at the top surface (top surface contains attached probes) of a substrate with this surface generating the second harmonic light. FIG. 12C depicts a similar apparatus to FIG. 12B except that the bottom surface (bottom surface contains attached probes) generates the second harmonic light.

FIG. 13 depicts two embodiments of an apparatus in which second harmonic light is generated by total internal reflection at an interface. The points of generation of the second harmonic light are denoted by the circles. In FIG. 13A, a dove prism is used to guide the light to a surface capable of generating the second harmonic light (bottom surface of prism but can also be another surface coupled to the prism through an index-matching material). In FIG. 13B, a wave-guide structure is used to produce multiple points of second harmonic generation.

FIG. 14 depicts three embodiments of an apparatus in which second harmonic light is generated using a fiber optic line (with attached probes at the end of the fiber). FIG. 14A depicts an apparatus in which both generation and collection of the second harmonic light occur in the same fiber. FIG. 14B depicts the use of a bead containing surface-attached probes at the end of the fiber. FIG. 14C depicts an apparatus in which the second harmonic light is generated at the end of the fiber optic (containing attached probges) and collected using a mirror or lens external to the fiber optic.

FIG. 15 depicts two embodiments of an apparatus using an optical cavity for power build-up of the fundamental.

FIG. 16 depicts three embodiments of an apparatus in which the mode of generation and collection of the second harmonic light uses reflection of the light from an interface.

### DESCRIPTION OF THE DRAWINGS

The drawings illustrate various embodiments of the apparatus and sample using second harmonic generation. The use of sum or difference frequency is not illustrated herein, but a similar set-up is required—with the use of two fundamental beams (ω₁,ω₂) where ω₁±ω₂=Ω, with Ω the sum or difference frequency. In the case where the sample surfaces are arrays comprised of discrete elements, a single element or more than one in parallel can be addressed with the fundamental light. Furthermore, detection can be made on a single element or many in parallel depending on the specific apparatus set-up.

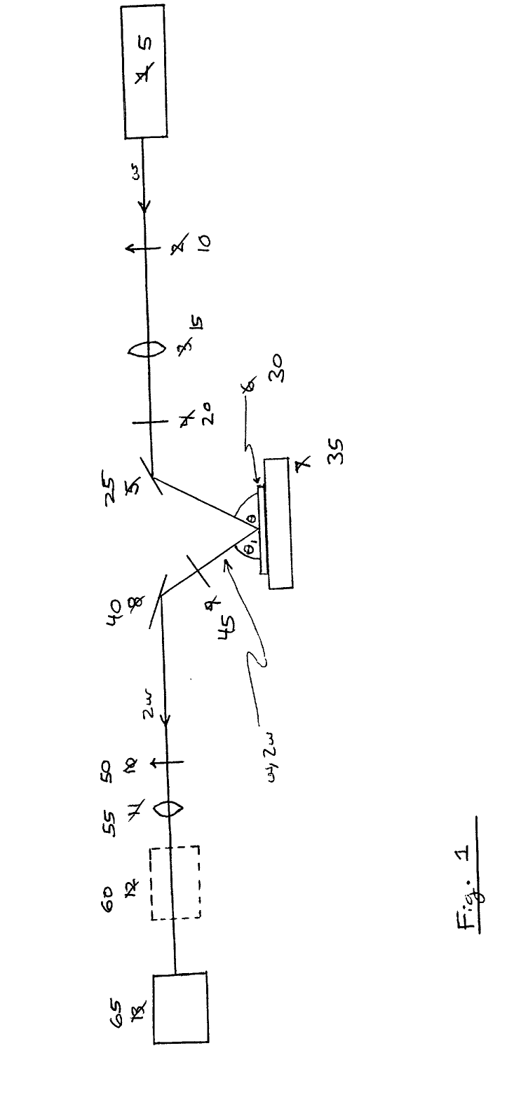

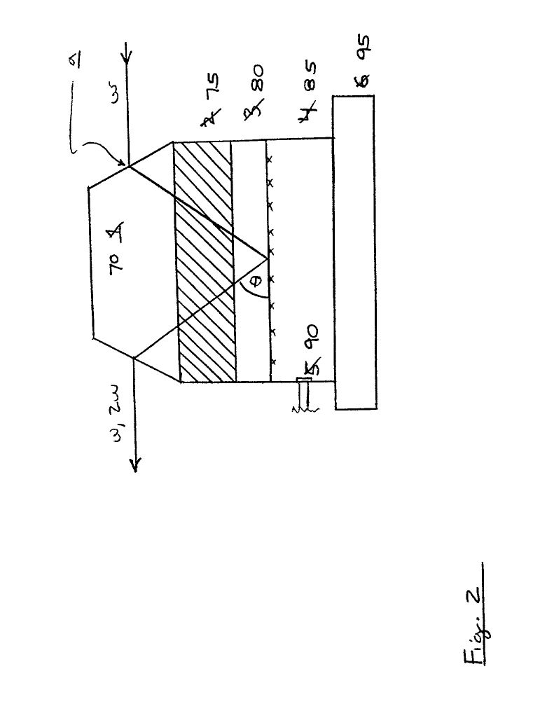

FIG. 1 illustrates an embodiment wherein second harmonic light is generated by reflecting incident fundamental light from the surface. Light source 5 provides the fundamental light necessary to generate second harmonic light at the sample. Typically this will be a picosecond or femtosecond laser, either wavelength-tunable or not tunable, and commercially available. Light at the fundamental frequency (ω) exits the laser and its polarization is selected using, for example a half-wave plate 10 appropriate to the frequency and intensity of the light (eg., available from Melles Griot, Oriel or Newport Corp.). The beam is then focused by lens 15 and passes through a pass filter 20 designed to pass the fundamental light but block the nonlinear light (eg., second harmonic). This filter is used to prevent back-reflection of the second harmonic beam into the laser cavity which can cause disturbances in the lasing properties. The beam is reflected from a mirror 25 and impinges at a specific location and with a specific angle θ on the surface. The mirror 25 can be scanned if required using a galvanometer-controlled mirror scanner, a rotating polygonal mirror scanner, a Bragg diffractor, acousto-optic deflector, or other means known in the art to allow control of a mirror's position. The sample surface 30 can be mounted on an x-y translation stage 35 (computer controlled) to select a specific location on the surface for generation of the second harmonic beam. The surface can be glass, plastic, silicon or any other solid surface which reflects the fundamental or second harmonic beams. The sample surface can be enclosed and the surface in contact with liquid. Furthermore, the sample 30 can be fed or drained by microcapillary or other liquid-transporting channels (not shown), pumps or electrophoretic elements, and these devices can be computer-controlled. The fundamental and the second harmonic outgoing beams (at specific angles with respect to the surface, i.e. θ₁—they are typically nearly colinear in direction) then reflected from the surface and the fundamental is filtered using a pass-filter 45 for the second harmonic beam, leaving only the harmonic beam (2ω). The second harmonic is reflected from mirror 40, its polarization selected if necessary by polarizing optic 50, and is focused using a lens 55 onto a detector 60. The lenses 15 and 55 can also be any combination of lenses known in the art for focusing or beam shaping. If required, a monochromator 60 can also be used to select a specific wavelength within the spectral band of the second harmonic beam. The detector can be a photomultiplier tube, a CCD array, or any other detector device known in the art for high sensitivity. For instance, a photomultiplier tube operated in single-photon counting mode can be used. At the detector, the light generates a voltage proportional to its intensity. Data is recorded for each location on the array surface as it is translated by the stage, scanned (or a combination thereof) and an image is built up of the second harmonic intensity generated from each region on the surface.

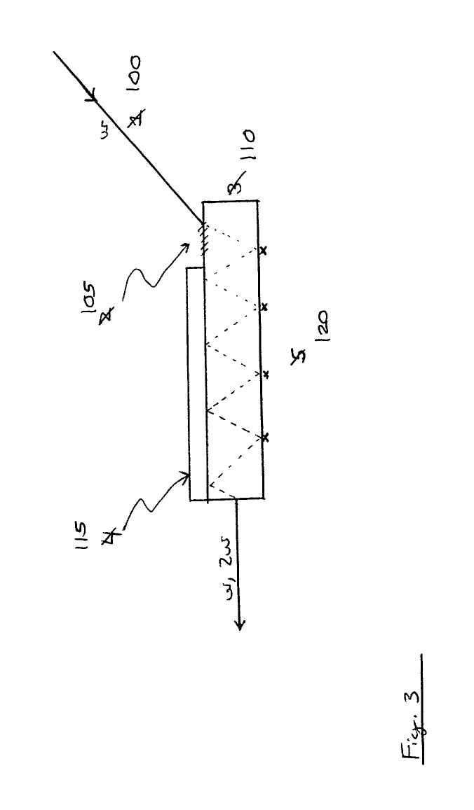

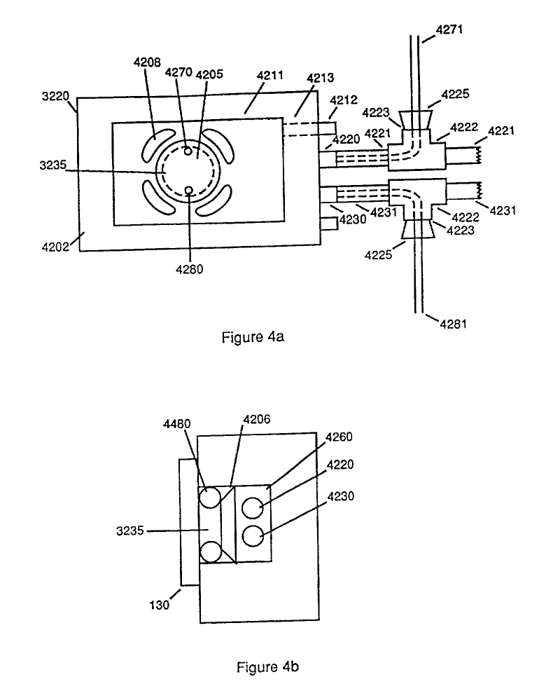

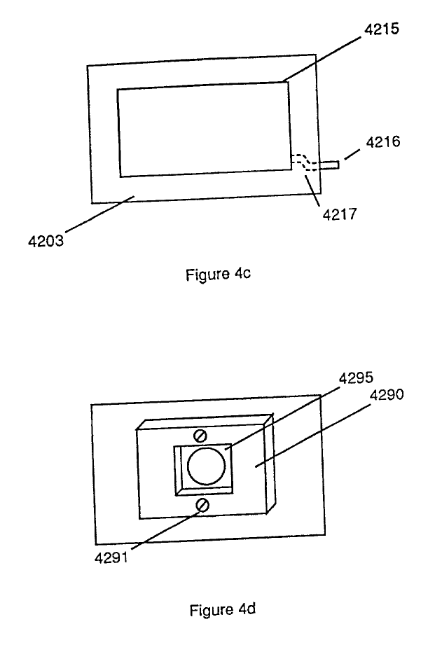

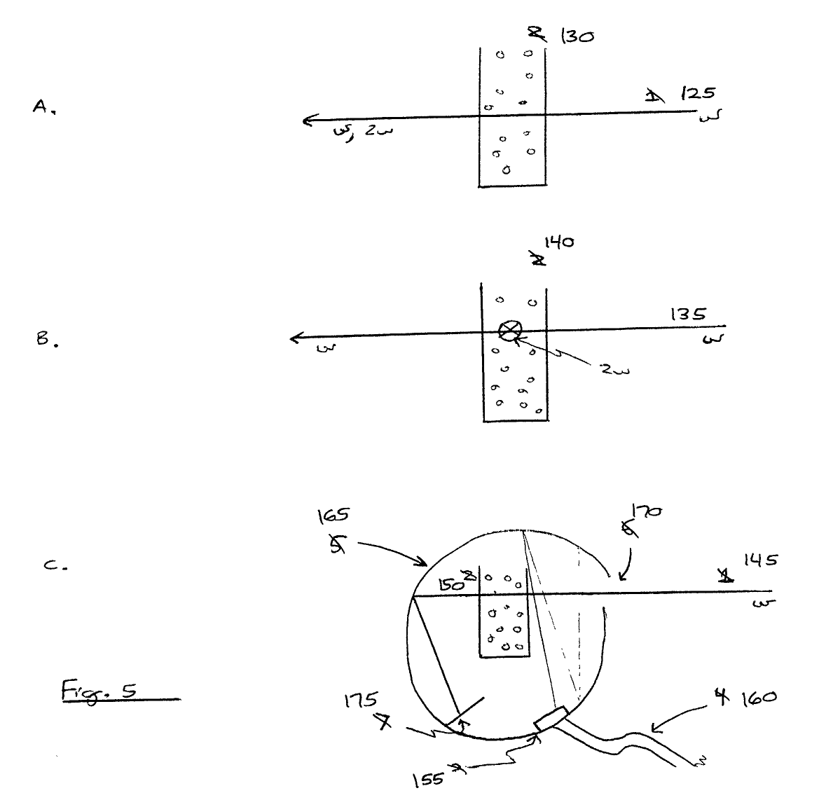

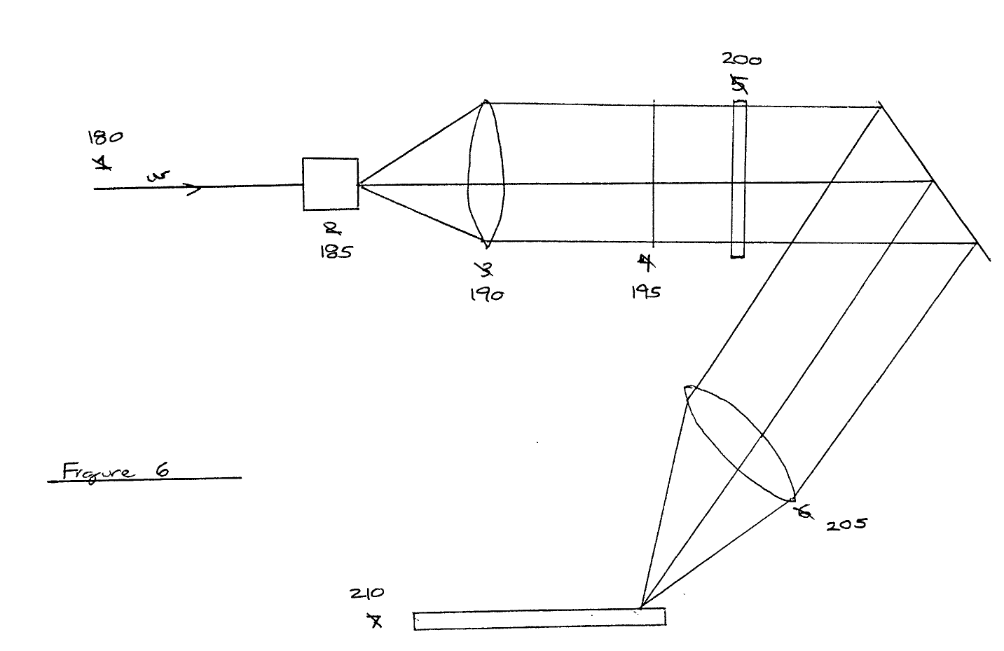

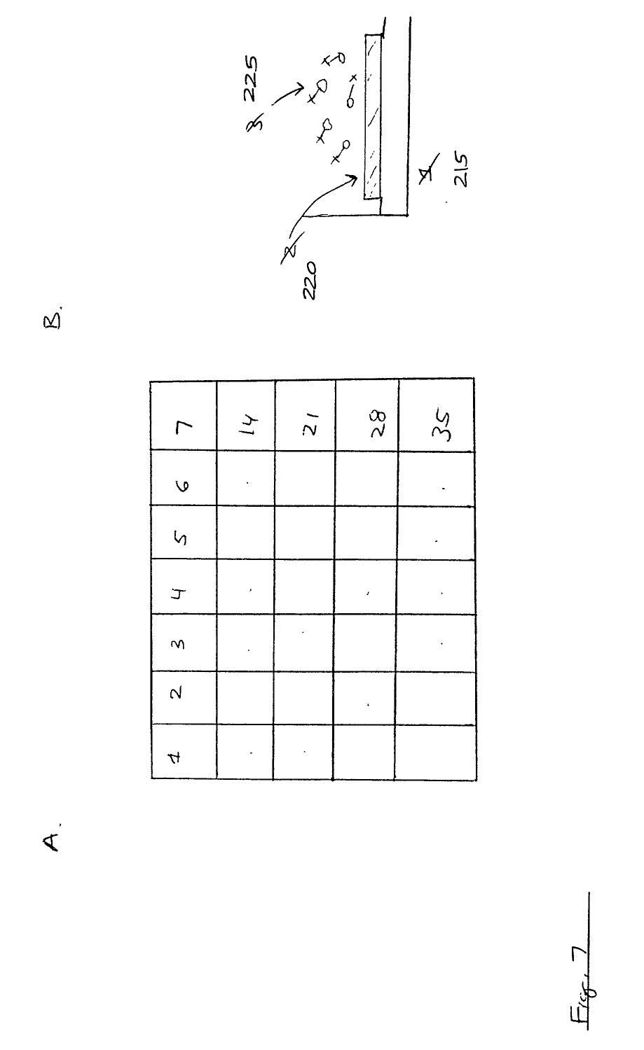

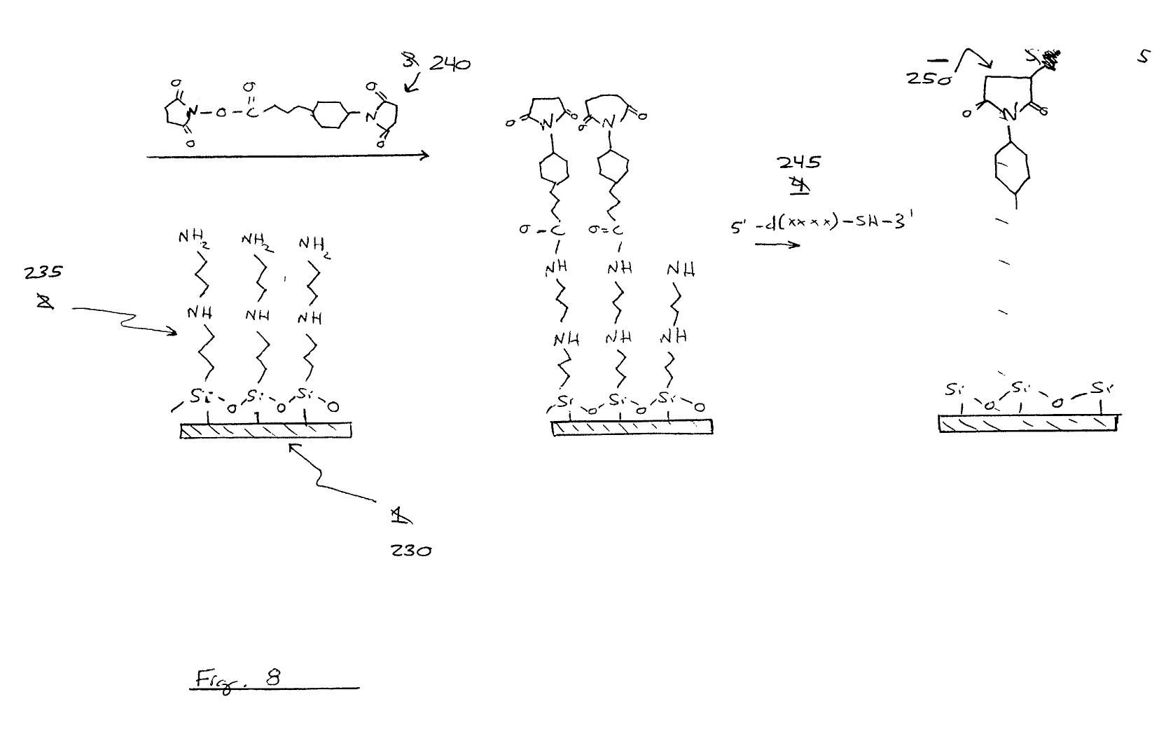

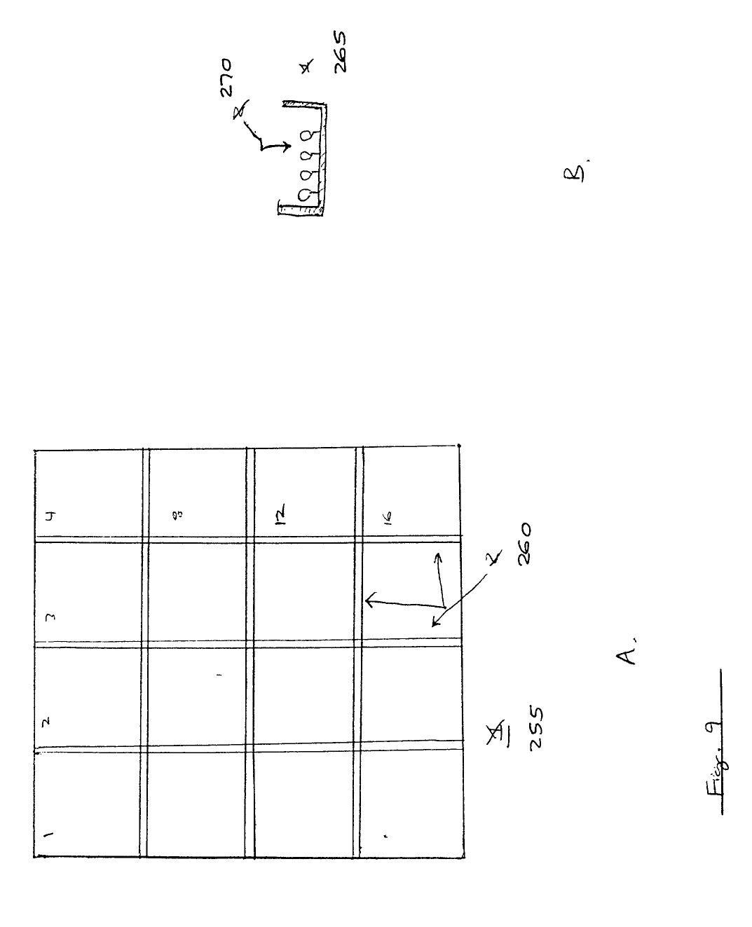

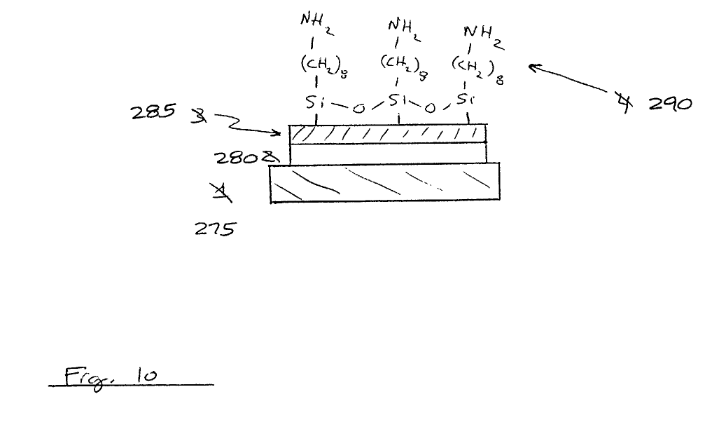

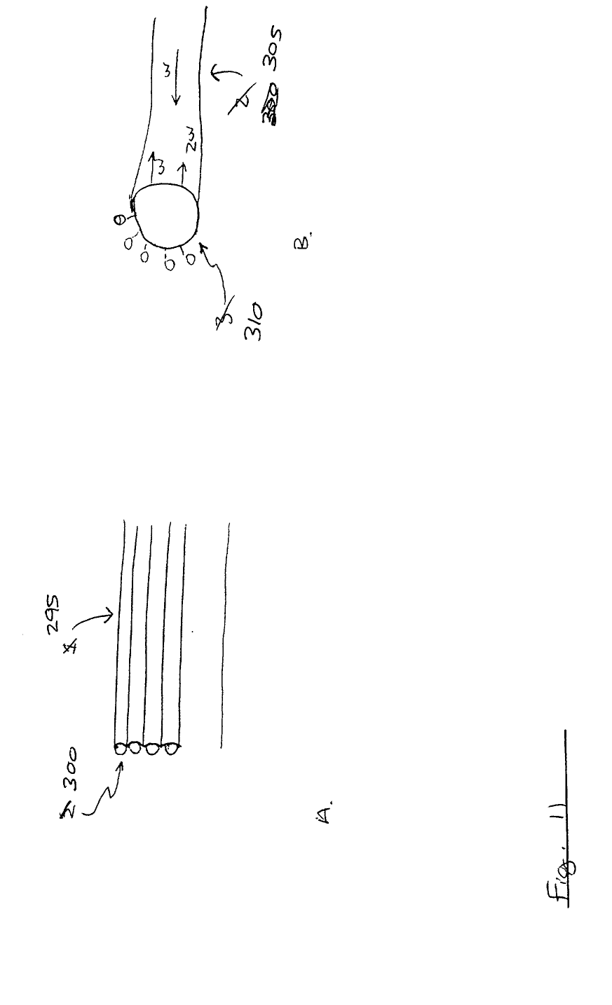

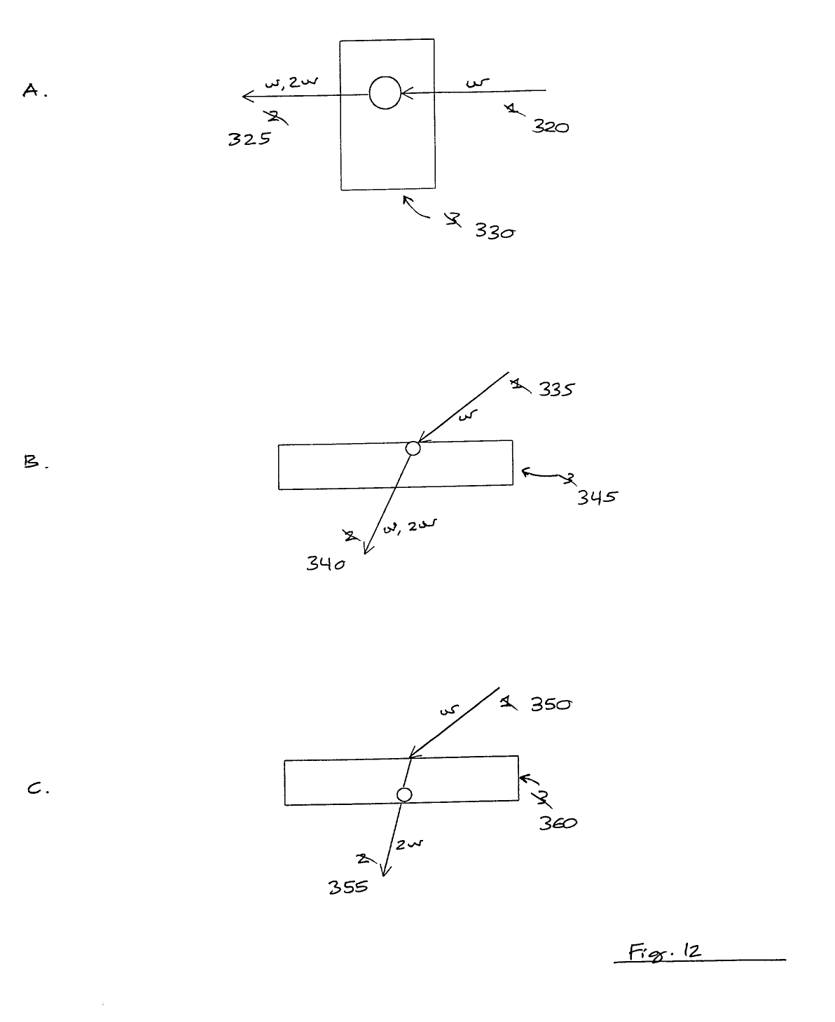

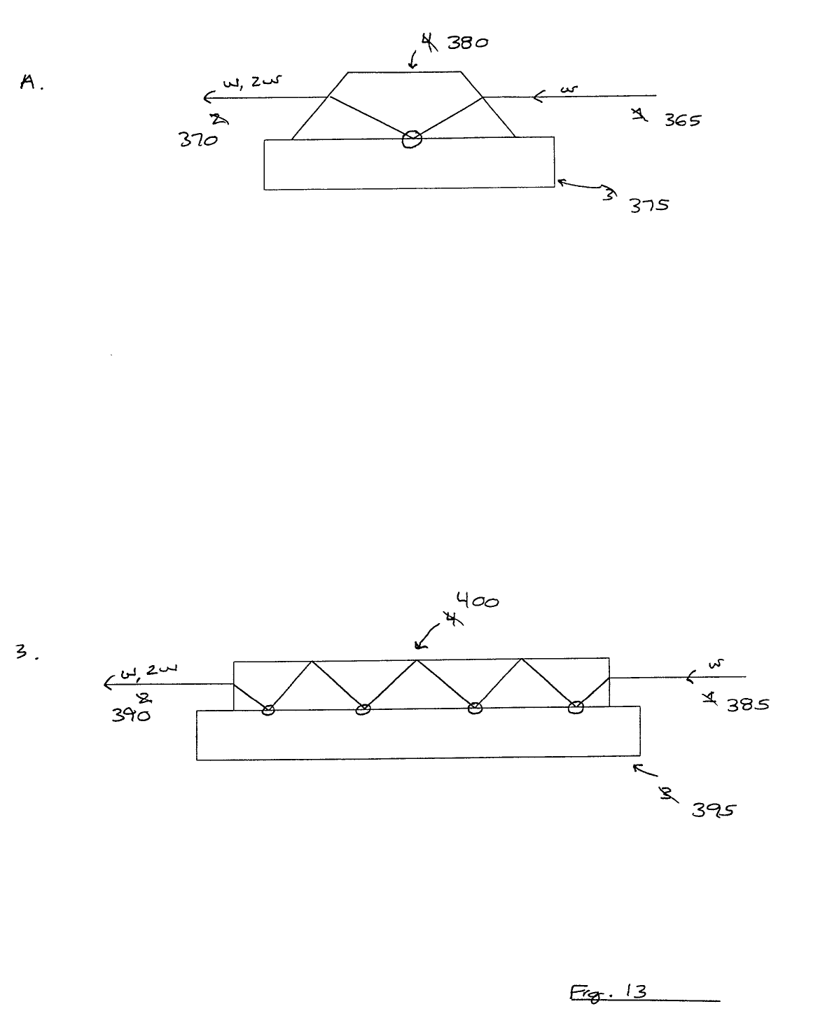

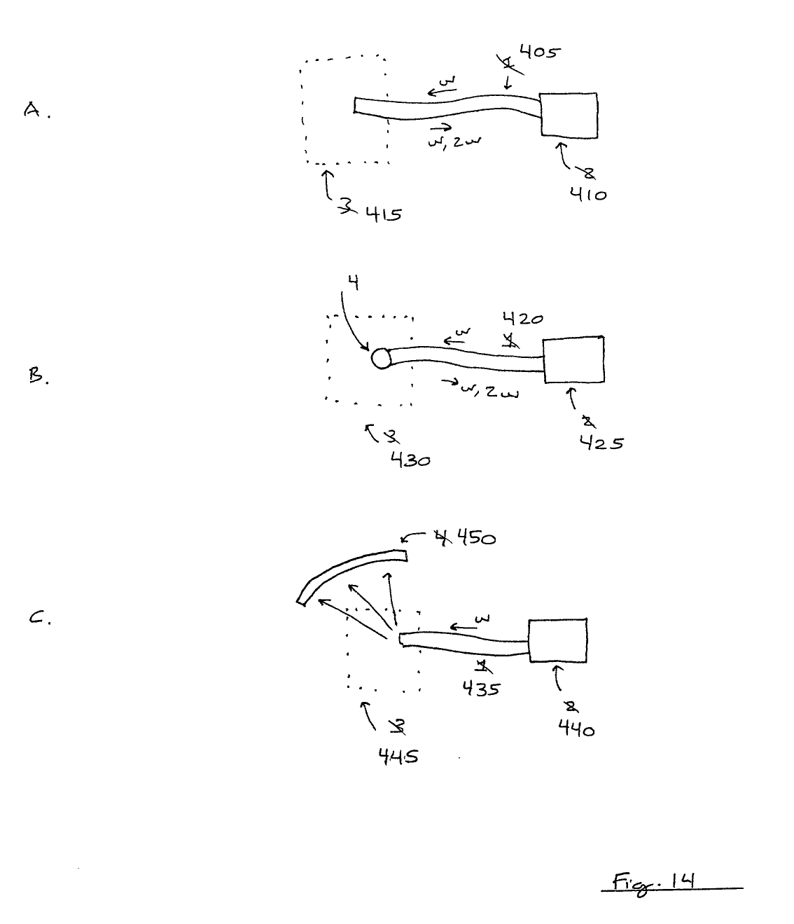

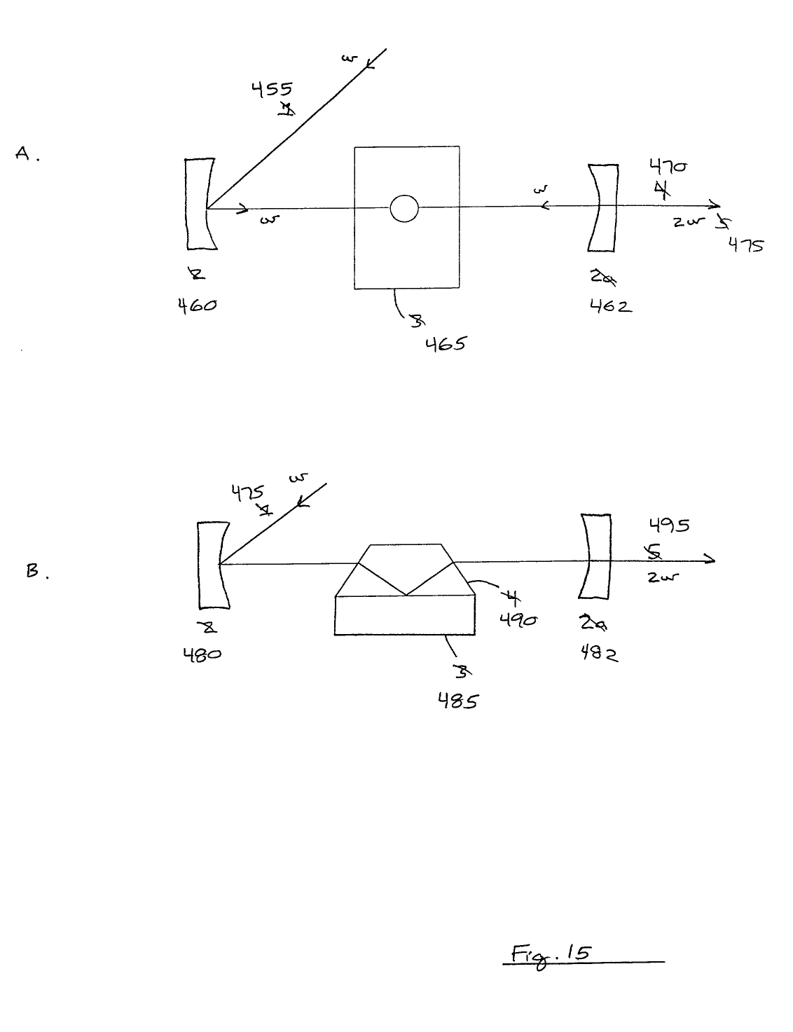

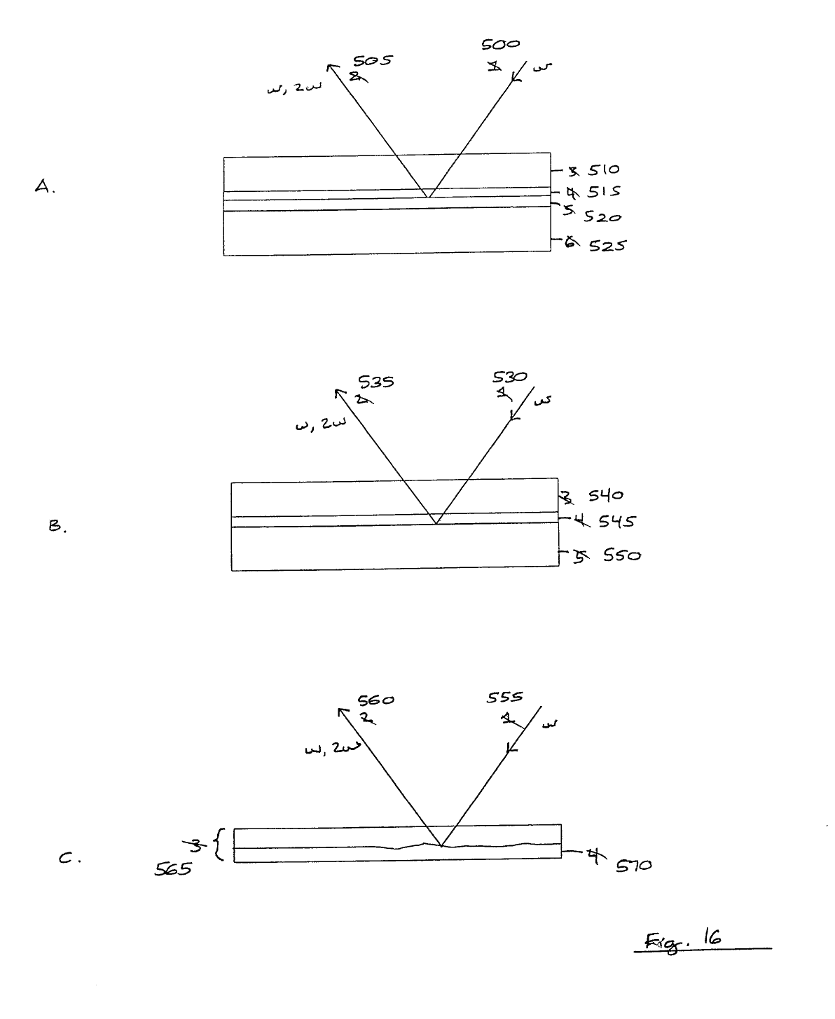

FIG. 2 illustrates an embodiment in which total internal reflection (evanescent wave generation) is used to generate the second harmonic light. Fundamental light (ω) is directed on to the surface of a prism element 70. The beam is refracted at position (a) and passes through the prism, through an index matching film 75 and impinges on substrate 80. Prism 70 and substrate 80 are made of optically transparent materials and are preferably of the same type. Prism 70 can be a Dove prism or any other element which can support evanescent fields (eg., waveguides, fibers and thin metallic films). The refractive index matching film 75 can be an oil, but is preferably a compressible optical polymer such as those disclosed by Sjodin, “Optical interface means”, PCT publication WO 90/05317, 1990. The prism 75 and the substrate 80 can also be a unitary, integral piece made of the same material (i.e, without the index matching film). An evanescent wave is generated at the interface between 80 and the medium in sample compartment 85 according to the indices of refraction in 80 and 85 and the angle of incidence of the beam at their interface. The electric field amplitude decays exponentially away from the substrate surface with a 1/e length ranging from nanometers to microns depending on several factors, including the surface electric potential, the counterion density in the sample compartment (if any). The sample compartment can be filled with air, a gas, or a liquid such as a solution or water. The ‘x’ marks on the surface of 80 facing the sample compartment emphasize that the sample of interest (eg., fabricated probes) are placed on this side. Substrate 80 can be a ‘chip’ which can be slid out between 75 and 85, allowing for measurement of different substrates. Element 90 in the drawing refers to a port in the sample compartment for drawing liquid or gases in and out of the compartment, for instance by pumps, electrostatic means, etc. The entire sample assembly can be mounted on an x-y translation stage 95 if necessary.

FIG. 3 illustrates an embodiment in which a slab-dielectric waveguide is used to deliver the fundamental light to the sample surface (the light beams are generated, directed and detected as in Drawing I with elements 1-5 and 8-13). A parallel plate or dielectric waveguide can be used to couple the fundamental light into a waveguide propagating mode. The drawing shows two slabs (110 and 115) and region (120). If the indices of refraction of slab 115 and region 120 are less than the index of refraction of the light (for both fundamental and second harmonic), a waveguiding mode can be developed. This mode produces multiple internal reflections at the substrate which can be used to increase the amount of second harmonic light generated by the interface. The fundamental beam 100 can be coupled into the waveguide 110 using a diffraction grating 105 scribed or embossed on the top surface of the waveguide, for example. The fundamental is propagated along the length of the waveguide and makes multiple total internal reflections at the top and bottom surfaces. The ‘x’ marks on substrate 110 denote the surface sample to be measured (i.e., containing the probes). If this interface generates significantly more second harmonic light than the interface between materials 110 and 115, the light intensity can be neglected. For example, if SH-labeled targets are bound to immobilized probes at the ‘x’ locations and the atomic structure at the interface between 110 and 115 is epitaxially matched, the interface 110/120 will generate much more second harmonic light than the interface 110/115.

The scope of the invention should, therefore, be determined not with the reference to the above description, but should instead be determined with reference to the appended claims, along with the full scope of equivalents to which such claims are entitled.

References
U.S. Patent Documents

| | | |
|---|---|---|
| 5,324,591 | Jun., 1994 | Georger, Jr. et al. |
| 5,215,899 | Jun., 1993 | Dattagupta |
| 6,030,787 | Feb., 2000 | Livak et al. |
| 5,834,758 | Nov., 1998 | Trulson et al. |
| 6,025,601 | Feb., 2000 | Trulson et al. |
| 5,631,734 | May, 1997 | Stern et al. |
| 6,084,991 | July, 2000 | Sampas |
| 5,633,724 | May, 1997 | King et al. |
| 6,121,983 | Sept., 2000 | Fork et al. |
| 5,485,277 | Jan., 1996 | Foster et al. |
| 5,324,633 | June, 1994 | Fodor et al. |
| 6,124,102 | Sept., 2000 | Fodor et al. |
| 5,847,400 | Dec., 1998 | Kain et al. |
| 5,432,610 | July, 1995 | King et al. |
| 5,320,814 | June, 1994 | Walt et al. |
| 5,250,264 | Oct., 1993 | Walt et al. |
| 5,298,741 | March, 1994 | Walt et al. |
| 5,252,494 | Oct., 1993 | Walt et al. |
| 6,023,540 | Feb., 2000 | Walt et al. |
| 5,814,524 | Sept., 1998 | Walt et al. |
| 5,244,813 | Sept., 1993 | Walt et al. |
| 5,512,490 | April, 1996 | Walt et al. |
| 6,095,555 | July, 2000 | Fiekowsky et al. |
| 6,134,002 | Oct., 2000 | Stimson et al. |
| 6,110,426 | Dec., 1997 | Shalon et al. |
| 6,040,586 | March, 2000 | Slettnes et al. |

References:

1. J. S. Salafsky, K. B. Eisenthal, “Second Harmonic Spectroscopy: Detection and Orientation of Molecules at a Biomembrane Interface”, Chemical Physics Letters 2000, 319, 435-439.

2. J. S. Salafsy, K. B. Eisenthal, “Protein Adsorption at Interfaces Detected by Second Harmonic Generation”, Journal of Physical Chemistry B, 2000, 104, 7752-7755.

3. R. Ekins, F. W. Chu, Trends in Biotechnology, 1999, 17, 217.

4. G. MacBeath and S. L. Schreiber, “Printing Proteins as Microarrays for High-Throughput Function Determination”, Science 2000, 289, 1760-1763.

5. N. J. Harrick, “Internal Reflection Spectroscopy”, John Wiley & Sons, Inc., New York, 1979.

6. Groves, J. T., Ulman, N., Boxer, S. G., “Micropatterning fluid bilayers on solid supports”, Science, 1997, 275, 651-3.

7. Fodor et al., “Light-directed Spatially-addressable Parallel Chemical Synthesis,” Science, 1991, 251, 767-773.

8. Ramsay, G. DNA chips—states-of-the-art. Nature Biotechnology 1998, 16(1), 40-44.

9. Marshall, A.; Hodgson, J. DNA chips—an array of possibilities. Nature Biotechnology 1998, 16(1), 27-31.

10. S. C. Case-Green et al., Curr. Opin. Chem. Biol. 2 (1998), 404.

11. S. A. Fodor, Science 277 (1997), 393.

12. D. H. Duggan et al., Nature Genet. 21 (Suppl.) (1999), 10.

13. M. Schena et al., Science 270 (1995), 467.

41. P. O. Brown and D. Botstein, Nature Genet. 21 (Suppl.) (1999), 33.

42. L. McAllister et al., Am. J. Hum. Genet. 61 (Suppl.) (1997), 1387.

16. A. P. Blanchard et al., Biosens. And Bioelectron. 11 (1996) 687.

17. G. J. Zhang et al., Anal. Left. 32, 2725 (1999).

18. F. Kleinjung et al., Anal. Chim. Acta 350, 51 (1997).

19. A. H. Uddin et al, Nucleic Acids Res. 25, 4139 (1997).

20. P. A. E. Piunno et al., Anal. Chem. 67, 2635 (1995).

21. F. F. Bier et al., Sens. Actuators B Chem. 38, 78 (1997).

22. A. P. Abel et al., Anal. Chem. 68, 2905 (1996).

23. J. A. Ferguson et al., Nature Biotechnol. 14, 1681 (1996).

24. P. Pantano and D. R. Walt, Chem. Mater. 8, 2832 (1996).

25. K. L. Michael et al., Anal. Chem. 70, 1242 (1998).

26. R. J. Fulton et al., Clin. Chem. 443, 1749 (1997).

27. M. Chee and D. R. Walt, unpublished results.

28. F. J. Steemers et al., Nature Biotechnol. 18, 91 (2000).

29. N. P. Gerry et al., J. Mol. Biol. 292, 251 (1999).

30. J. Liu and W. H. Tan, Anal. Chem. 71, 5054 (1999).

31. D. R. Walt, Science 287, (2000) 451.

[14] “Nonlinear optical measurement of membrane potential around single molecules at selected cellular sites”, G. Peleg, A. Lewis, M. Linial, L. M. Loew, Proc. Natl. Acad. Sci. V. 96, 1999, 6700-6704.

[15] G. Boyd, T. Rasing, R. Leite, Y. Shen (1984) Phys. Rev. B 30, 519-526.

[16] C. Chen, T. F. Heinz, D. Richard, Y. Shen (1981) Phys. Rev. Lett. 46, 1010-1012. Milan Mrksich and George M. Whitesides, “Using Self-Assembled Monolayers to Understand the Interactions of Man-Made Surfaces with Proteins and Cells,” Annu. Rev. Biophys. Biomol. Struct., 1996, 25:55-78.

V. G. Cheung et al., “Making and reading microarrays”, Nature Genetics (Suppl.), 1999, 21: 15-19.

L. A. Chrisey, G. U. Lee, C. E. O'Ferrall, “Covalent attachment of synthetic DNA to self-assembled monolayer films”.

Gabriel P. Lopez, Mark W. Albers, Stuart L. Schreiber, Reed Carroll, Ernest Peralta and George M. Whitesides, Convenient Methods for Patterning the Adhesion of Mammalian Cells to Surfaces Using Self-Assembled Monolayers of Alkanethiolates on Gold, J. Am. Chem. Soc., 1993, 115, 5877-5878.

Rahul Singhvi, Amit Kumar, gabriel P. Lopez, Gregory N. Stephanopoulos, Daniel I. C. Wang, George M. Whitesides and Donald E. Ingber, “Engineering Cell Shape and Function,” Science, vol. 264, Apr. 29, 1994, 696-698.

Levicky R, Herne T M, Tarlov M J, and Satija S K (1998). “Using Self-Assembly to Control the Structure of DNA Monolayers on Gold: A Neutron Reflectivity Study.” Journal of the American Chemical Society 120: 9787-9792.

1. A method for measuring an interaction at an interface between an attached probe and a labelled target, said method comprising measuring an effect of said interaction between said attached probe and said labeled target at said interface using a surface-selective nonlinear optical technique.

2. The method of claim 1 wherein said attached probe is coupled or conjugated in-vitro to a substrate or solid surface.

3. The method of claim 1, wherein said probe comprises or is part of a surface selected from the group consisting of biological cells, liposomes, vesicles, beads, particles.

4. The method of claim 1 wherein said probe is patterned on a substrate or solid surface.

5. The method of claim 1, wherein said probes is patterned in an array format on a substrate or solid surface.

6. The method of claim 1, wherein said probe is comprised of oligonucleotides or polynucleotides of DNA or RNA, said oligonucleotides possessing a particular base-pair sequence, with said sequence attached to a specific region location on a solid surface or substrate.

7. The method of claim 6, wherein the sequences of the oligonucleotides are patterned in a microarray format.

8. The method of claim 6, wherein said oligonucleotides are attached to regions on the surface of size nanometers to microns in dimension.

9. The method of claim 1, wherein said attached probe is comprised of protein possessing a particular amino-acid sequence, with said proteins attached to a specific region on a solid surface or substrate.

10. The method of 1, wherein said probe comprises proteins patterned in a microarray format.

11. The method of claim 1, wherein said probe is selected from the group consisting of nucleic acid, protein, small molecule, organic molecule, biological cell, virus, liposome, receptor, antibody, agonist, antagonist, inhibitor, ligand, antigen, oocyte, hormone, protein, peptide, receptor, drug, enzyme, nucleoside, carbohydrate, cDNA, oligonucleotide, polynucleotide, oligosaccharide, peptide nucleic acid (PNA), toxin, nucleic acid analog, ion channel receptor, said probes patterned in an array format on a substrate or solid surface, with the properties or chemical identity of said probes remaining constant or varying among regions comprising said array.

12. The method of claim 11, wherein said probe of a given base-pair sequence is attached to regions on the surface of size nanometers in dimension.

13. The method of claim 9, wherein said protein is attached to regions on the surface of size nanometers in dimension.

14. The method of claim of 4, wherein said attached probes are attached in a plurality of known regions which comprise an array or microarray.

15. The method of claim 1, wherein the nonlinear optical technique is selected from the group consisting of second harmonic, sum frequency or difference frequency generation.

16. The method of claim 1, wherein the mode of generation, collection or detection of the nonlinear optical radiation uses one or more modes selected from the group consisting of reflection, transmission, evanescent wave, multiple internal reflection, near-field optical techniques, confocal, optical cavity, planar waveguide, fiber-optic and dielectric-slab waveguide, near-field techniques.

17. The method of claim 1 wherein said technique comprises measuring a change in nonlinear optical radiation emitted from said interface.

18. The method of claim 1 wherein said technique comprises measuring a change in nonlinear optical radiation emitted from said interface.

19. The method of claim 18 wherein said change in nonlinear optical radiation is due to an increase or decrease in labeled targets at an interface.

20. A method for studying the degree or extent of binding of an attached probe and a labeled target at an interface utilizing a surface selective nonlinear optical technique comprising measuring the effect said binding has on said labeled target at said interface.

21. The method using a surface selective nonlinear optical technique wherein targets or decorators coupled to labels are used to detect probe-target binding reactions, and wherein the nonlinear optical properties or hyperpolarizability of said labels can be changed by an agent or light beam acting as a trigger.

22. The method of claim 21, wherein said labels are caged or are molecular beacons.

23. The method of claim 21, wherein ultraviolet light acts to cleave a bond between a nonlinear active moiety in said labels and a second moiety.

24. The method of 1, wherein said optical technique determines nonlinear light intensity by measuring the intensity of the nonlinear light at a region or plurality of regions over a period of time.

25. The method of 1, wherein said optical technique determines the nonlinear light intensity by measuring the intensity of the nonlinear light at a region or plurality of regions with varying target concentration.

26. The method of 1, wherein said probes are attached to a metal surface, semiconductor surface, glass surface, a latex surface, a solid surface, a substrate, a gel substrate, a fiber-optic surface, a silica surface or a bead surface.

27. The method of claim 26 wherein the surface is chemically derivatized.

28. The method of claim 27 wherein said surface is derivatized with a self-assembled monolayer or with an organosilane.

29. The method of claim 1, wherein said probes are attached to a planar or non-planar surface.

30. The method of claim 1, wherein said reactions between attached probes and labeled targets include one or more biological component selected from the group consisting of nucleic acid, ligand, protein, small molecule, organic molecule, biological cell, virus, liposome, receptor, agonist, inhibitor, antibody, antigen, peptide, oocyte, hormone, drug, enzyme, ligand, carbohydrate, hapten, nucleoside, oligosaccharide, organic molecule, toxin, oligonucleotide, polynucleotide, hormone, nucleic acid analog, peptide nucleic acid (PNA), cDNA, ion channel receptor.

31. The method of claim 1, wherein said labeled target is one or more of the following components: a nucleic acid, protein, small molecule, organic molecule, biological cell, virus, liposome, receptor, antibody, agonist, antagonist, inhibitor, hapten, ligand, antigen, oocyte, hormone, protein, peptide, receptor, drug, enzyme, nucleoside, carbohydrate, cDNA, oligonucleotide, nucleoside, polynucleotide, oligosaccharide, peptide nucleic acid (PNA), toxin, nucleic acid analog, ion channel receptor.

32. The method of claim 1, wherein said attached probe is one or more of the following components: a nucleic acid, protein, small molecule, organic molecule, biological cell, oocyte, virus, liposome, receptor, antibody, agonist, antagonist, inhibitor, hapten, ligand, antigen, hormone, protein, peptide, receptor, drug, enzyme, nucleoside, carbohydrate, cDNA, oligonucleotide, nucleoside, polynucleotide, oligosaccharide, peptide nucleic acid (PNA), toxin, nucleic acid analog, ion channel receptor.

33. The method of claim 1, wherein said probes are attached to solid surfaces or are cells cultured on solid surfaces.

34. The method of claim 1, wherein one or more probes and targets are measured in reactions which occur at one or more surface regions over the same or many periods of time.

35. The method of claim 1, wherein the probe is an ion-channel receptor and the targets are signalling molecules, antagonists, agonists, gating molecules, drugs, neuropeptides or other compounds which induce or modulate channel behavior.

36. The method of claim 1, wherein one or more targets, agonists, antagonists, drugs or small molecules are used in combination with said probes and target and can be introduced to the sample before, during or after the time in which probe-target interactions occur.

37. The methods of claim 1, wherein the probe is an ion-channel receptor and the targets are signalling molecules, antagonists, agonists, gating molecules, drugs, neuropeptides or other compounds which induce or modulate opening and closing of said channel receptors.

38. The method of claim 1, wherein said reactions between said probes and targets comprise a probe-target binding reaction.

39. The method of claim 1, wherein said reactions are performed in the presence of a inhibitor selected from the group comprising: small molecules, drugs, agonists, blocking agents, or other components, said inhibitor affecting the probe-target binding process.

40. The method of claim 1, wherein said probe is covalently or non-covalently attached to a surface.

41. The method of claim 1, wherein said probe is attached to a self-assembled monolayer.

42. The method of claim 28, wherein the self-assembled monolayer is in the chemical family of silanes or terminal-functional silanes.

43. The method of claim 1, wherein said attached probe is a biological component and is reacted with said target to produce a mutual interaction.

44. The method of claim 1, where the thermodynamic or kinetic properties of said target-probe reactions are measured.

45. The method of claim 43, wherein the mutual interaction is a chemical bond, an electrostatic force, physisorption, chemical affinity, chemisorption, molecular recognition, physico-chemical binding, hydrogen bond or hybridization process.

46. The method according to claim 2, wherein said substrate or solid surface supports a phospholipid or artifical bilayer membrane.

47. The method according to claim 46, wherein said phospholipid or artificial bilayer comprises membrane proteins.

48. The method of claim 1, wherein said probes are attached to a surface comprising one or more of the following materials selected from the group: silica, polystyrene, metal, semiconductor, glass, silicon, silicon nitride, nylon, quartz and mixtures thereof.

49. The method of claim 1, wherein probes, targets, biological components or reagents are delivered to said interface, a solid surface, an array on the surface, or specific elements within said array using microfluid channels, electrophoresis or capillary electrophoresis.

50. A method of detecting a biological binding process at an interface between an attached probe and a target, said method comprising measuring the change in amount or orientation of labeled targets near the interface during the time said probe and said target are binding, said method of measuring comprising the steps of:
a. optionally measuring the background non-linear signal at the interface before binding; and
b. measuring the non-linear signal which is produced at the interface during the time said probe and said target are in the process of binding.

51. A method of detecting a biological binding process at an interface between an attached probe and a target, said method comprising measuring the change in amount or orientation of labeled targets near the interface during the time said probe and said target are binding, said method of measuring comprising the steps of:
a. optionally measuring the background non-linear signal at the interface before binding; and
b. measuring the non-linear signal which is produced at the interface after said probe has bound to said target.
c. Optionally increasing the concentration of said target and measuring the non-linear signal produced to determine the effect of concentration on probe/target binding.

52. A method of detecting the effect a potential inhibitor, agonist, antagonist, drug has on a biological binding process at an interface between an attached probe and a labeled target, said method comprising measuring the change in amount or orientation of labeled targets near the interface during the time said probe and said target are binding, said method of measuring comprising the steps of:
a. optionally measuring the background non-linear signal at the interface before binding; and
b. measuring the non-linear signal which is produced at the interface during the time said probe and said target are in the process of binding in the absence of said inhibitor, said agonist, said drug or said antagonist
c. measuring the non-linear signal which is produced at the interface during the time said probe and said target are in the process of binding in the presence of said inhibitor, agonist, antagonist or other compound.
d. Optionally increasing the concentration of said target and measuring the non-linear signal produced to determine the effect of concentration on probe/target binding.

53. The methods according to claims 50 further comprising the step of increasing the concentration of said target or said agonist, said antagonist, said drug or said inhibitor and measuring the non-linear signal produced to determine the effect of concentration on probe/target binding.

54. The method of claim 1 in which the polarization of the fundamental, second harmonic, sum frequency or difference frequency radiation beams can be adjusted in order to measure different orientational sub-populations of probes, targets, water molecules or indicators at the interface.

55. The method of claim 54 wherein the fundamental or nonlinear radiation is circularly polarized.

56. The method of claim 1, wherein the interface comprises a cell, liposome or vesicle surface or a solid surface or a substrate.

57. An apparatus for detecting reactions at an interface between attached probes and targets, or secondary reactions caused by said reactions, said apparatus comprising:
An optical source generating an electromagnetic wave or radiation beam, at a predetermined frequency or wavelength band;
A substrate with attached said probe;
Optional first optics between said optical source and said substrate for directing and scanning a beam of optical radiation onto said substrate at a predetermined angle.
An optical sensor; and
Optional second optics located between said substrate and said sensor, said second optics receiving radiation of predetermined frequency, emitted at a second angle relative to said substrate from said target and a probe attached thereto, said angle being predetermined, said radiation being emitted by said interface in response to said beam of laser radiation, said second optics directing nonlinear radiation to said sensor.

58. An apparatus for detecting reactions at an interface between attached probes and targets, or secondary reactions caused by said reactions, said apparatus comprising:
A substrate with attached said probe;
A source of optical radiation;
Optional first optics between a source of optical radiation and said substrate, said optics for directing and scanning a beam of optical radiation onto said substrate at a predetermined angle;
An optical detector; and
Optional second optics located between said substrate and said sensor, said second optics receiving radiation emitted at a second angle relative to said substrate from said target and a probe attached thereto, said angle being predetermined, said second optics directing radiation to said sensor.

59. The apparatus according to claim 57, wherein said second optics include a frequency selector element for isolating a predetermined frequency in the radiation received from said probe and said target.

60. The apparatus of claim 57 wherein said optical source is a laser which produces pulse trains, wherein each pulse is of duration of femtoseconds to nanoseconds.

61. The apparatus according to claim 57 wherein said second optics comprise an element to select radiation of a predetermined frequency approximately twice said first predetermined frequency.

62. The apparatus according to claim 57, wherein said predetermined frequency is a first predetermined frequency and said optical source is a first laser source, further comprising a second laser source generating an electromagnetic wave of said second predetermined frequency, said first optics including elements for directing an additional beam of laser radiation of said second predetermined frequency and for directing said additional beam to said probe and said target on said substrate.

63. The apparatus according to claims 57 wherein the radiation emitted from said probe and said target is due to a non-linear response, said predetermined frequency being selected to induce emission of the non-linear radiation from said probe and said target.

64. The apparatus according to claim 57 wherein most or all radiation emitted by said probe and said target in response to said beam of radiation of said predetermined frequency is emitted at said second predetermined angle.

65. The apparatus of claims 57 wherein said second optics allow for delivery or collection of said radiation to said interface using one or more of the following techniques: multiple internal reflection, near-field optical techniques, confocal, optical cavity, planar waveguide, fiber-optic and dielectric-slab waveguide, near-field techniques.

66. A method for measuring an interaction between an attached probe and a labelled target at an interface comprising one or more regions, said method comprising measuring an effect of said interaction between said attached probe and said labeled target at said interface using a surface-selective nonlinear optical technique.

67. The method of claim 66 wherein the probe-target reactions include an ion channel or receptor.

68. The method of claim 66 wherein the effects comprise an ion channel opening, closing or modulation.

69. A method for studying the degree or extent of binding of probes and targets at an interface in the presence of a decorator molecule or particle utilizing a surface selective nonlinear optical technique, said method comprising measuring the effect said binding has on said decorator molecule or particle.

70. The method of claim 69, wherein the decorator is a molecule or particle possessing a hyperpolarizability.

71. The method according to claim 69 wherein said interface is comprised of a surface and said probes are attached to said surface in one or more regions of an array.

72. The method of claim 69 wherein the decorator has a specific binding affinity for a target, a probe, a target-probe complex, or for other species, said species having a binding affinity for said target, said probe or said target-probe complex.

73. The method of claim 69 wherein the decorator molecule or particle is dissolved or suspended in a phase containing the target component at a concentration of about 1 picomolar to about 500 millimolar.

74. The method of claim 69, wherein said interface is comprised of a solid substrate, a solid surface, a cell surface or a liposome surface.

75. The method of claim 69, wherein said interface comprises a glass surface, a latex surface, a fiber-optic surface, a silica surface, a silicon surface, a porous silicon surface, a plastic surface or a bead surface, a cell surface or a liposome surface.

76. The method of claim 75 wherein said surface is chemically derivatized.

77. The method of claim 75 wherein said substrate is chemically derivatized with a self-assembled monolayer or an organosilane.

78. The method of claim 69, wherein said interface comprises a planar or non-planar surface.

79. The method of claim 69, wherein said probe or said target is a biological component selected from the group comprising: nucleic acid, protein, small molecule, organic molecule, biological cell, oocyte, virus, liposome, receptor, antibody, agonist, antagonist, inhibitor, hapten, ligand, antigen, hormone, protein, peptide, receptor, drug, enzyme, nucleoside, carbohydrate, cDNA, oligonucleotide, nucleoside, polynucleotide, oligosaccharide, peptide nucleic acid (PNA), toxin, nucleic acid analog, ion channel receptor.

80. The method of claim 69, wherein said probes, said targets or said decorator is a modulator selected from the group consisting of small molecules, drugs and blocking agents.

81. The method of claim 69, wherein said probe is covalently or non-covalently attached to a surface.

82. The method of claim 81, wherein said probe is covalently attached to said surface by a self-assembled monolayer.

83. The method of claim 82, wherein the self-assembled monolayer is in the chemical family of silanes or terminal-functional silanes.

84. The method of claim 79, wherein the attached biological component is reacted with a target for the purpose of studying the mutual interaction.

85. The method of claim 79, where binding has thermodynamic or kinetic properties which are measured.

86. The method of claim 79, wherein said binding of said probe and said target occurs through a chemical bond, an electrostatic force, physico-chemical binding, hydrogen bond or hybridization process.

87. The method of claim 69, wherein said target is selected from the group consisting of a nucleic acid, protein, small molecule, biological cell, virus, liposome, receptor, agonist, antagonist, inhibitor, hormone, antibody, antigen, peptide, receptor, drug, enzyme, ligand, nucleoside, polynucleoside, carbohydrate, cDNA, hormone, allergen, cDNA, hapten, oligonucleotide, biotin, streptavidin, polynucleotide, oligosaccharide, peptide nucleic acid (PNA) and nucleic acid analog.

88. The method of claim 69, wherein the mode of generation, collection or detection of the nonlinear optical waves uses one or more modes selected from the group consisting of reflection, transmission, evanescent wave, multiple internal reflection, near-field optical techniques, confocal, optical cavity, planar waveguide, fiber-optic and dielectric-slab waveguide, near-field techniques.

89. The method of claim 74, wherein said solid substrate is a solid, planar support or nanometer- or micron-sized beads.

90. The method of claim 69, wherein said probe or said target is attached to a substrate or solid surface.

91. The method of claim 69, wherein said probe or target is patterned in a two-dimensional array on said substrate or solid surface.

92. The method of claim 69, wherein said probe or said targets are delivered to a solid surface, an array on the surface, or specific elements within said array using microfluid channels or capillary electrophoresis.

93. The method of claim 91, wherein said surface supports a phospholipid bilayer.

94. The method of claim 69, wherein biological cells are attached to or patterned on a substrate or solid substrate.

95. The method of claim 69, wherein said target is a drug or blocking agent.

96. A method for measuring an adsorption process of a labelled target to an interface or solid surface, said method comprising measuring an effect of said adsorption using a surface-selective nonlinear optical technique.

97. The method of claim 38, wherein said binding is a nucleic acid hybridization, wherein said probe and target components are nucleic acids, oligonucleotides, RNA or DNA.

98. The method of claim 69 wherein said probe and target are peptides or proteins.

99. The method of claim 69, wherein said probe is a cell surface and said target is a virus binding to said cell surface.

100. The method of claim 69, wherein the proteins or peptides are genetically engineered or selected to bind a decorator molecule or particle.

101. A method of detecting reactions at an interface between an probe and a labeled target, said method comprising measuring the effect said binding of said target has on a nonlinear-signal generated by a decorator molecule or particle, said decorator having selective affinity for said target, said probe or a target-probe complex, said method of measuring comprising the steps of
a. optionally measuring the background non-linear signal at the interface before binding; and
b. measuring the non-linear signal which is produced at the interface during the time said probe and said target are in the process of binding.
c. Optionally increasing the concentration of said target and measuring the non-linear signal produced to determine the effect of concentration on probe/target binding.

102. A method of detecting reactions at an interface between an attached probe and a target, said method comprising measuring the effect said binding of the target has on the amount of nonlinear-signal generated by a decorator molecule or particle, said decorator having selective affinity for said target, said probe or a target-probe complex resulting from said binding process, said method comprising the steps of
a. optionally measuring the background non-linear signal at the interface before binding; and
b. measuring the non-linear signal which is produced at the interface after said probe has bound to said target.
c. Optionally increasing the concentration of said target and measuring the non-linear signal produced to determine the effect of concentration on probe/target binding.

103. A method of detecting the effect a potential inhibitor, agonist, drug or antagonist has on reactions at an interface between an attached probe and a target, said method comprising measuring the effect said binding of the target has on the amount of nonlinear-signal generated by a decorator molecule or particle, said decorator having selective affinity for said target, said probe or a target-probe complex resulting from said binding process, said method comprising the steps of
a. optionally measuring the background non-linear signal at the interface before binding;
b. measuring the non-linear signal which is produced at the interface during the time when said probe and said target are in the process of binding in the absence of said inhibitor, said antagonist, said agonist or said drug and
c. measuring the non-linear signal which is produced at the interface during the time said probe and said target are in the process of binding in the presence of said inhibitor, said antagonist, said agonist or said drug.
d. Optionally increasing the concentration of said target and measuring the non-linear signal produced to determine the effect of concentration on probe/target binding.

104. The method according to claim 101 further comprising the step of increasing the concentration of said target and measuring the non-linear signal produced to determine the effect of concentration on probe/target binding.

105. The method according to claims 101 further comprising the step of increasing the concentration of said target or said agonist, said antagonist, said drug or said inhibitor and measuring the non-linear signal produced to determine the effect of concentration on probe/target binding.

106. The method of claim 101, wherein said probe or said target components or both said probe or said target are peptide nucleic acids (PNAs) or other nucleic acid analog.

107. The method of claim 101, wherein the decorator molecule or particle is present during the probe-target binding reaction or is added after said binding occurs.

108. The method of claim 101, wherein said decorator molecule or particle has a binding affinity for said target, said probe, or said target-probe complex.

109. The method of claim 101, wherein the decorator molecule or particle includes a biological component, a nucleic acid, protein, small molecule, biological cell, virus, liposome, receptor, agonist, antagonist, inhibitor, hormone, antibody, antigen, peptide, receptor, drug, enzyme, ligand, nucleoside, polynucleoside, carbohydrate, cDNA, hormone, allergen, cDNA, hapten, oligonucleotide, biotin, streptavidin, polynucleotide, oligosaccharide, peptide nucleic acid (PNA), nucleic acid analog.

110. The method of claim 101, wherein said binding is determined by measuring nonlinear the light intensity at a region or plurality of regions over a period of time.

111. The method of claim 101, wherein said binding is determined by measuring the nonlinear light intensity at a region or plurality of regions with varying target concentration.

112. The method of claim 101, wherein said probes and targets are nucleic acids or nucleic acid analogs, and said decorator possesses a selective affinity for either the probes, the target or their bound complex.

113. The method of claim 45, wherein said affinity is due to an intercalation process, a hydrogen bond, an electrostatic interaction, or some combination thereof.

114. The method of claim 101, wherein said decorator includes a moiety in the family of or inclusive of: psoralen, ethidium bromide, methanphosphonate, phosphoramidates, propidium iodide, acridine, 9-aminoacridine, acridine orange, chloroquine, pyrine, echinomycin, 4′,6-diamidino-2-phenylindole, dihydrochloride (DAPI), Succinimidyl acridine-9-carboxylate, chloroquine, pyrine, echinomycin, 4′,6-diamidino-2-phenylindole, dihydrochloride (DAPI), single-strand binding protein (SSB), tripyrrole peptides, flavopiridol, pyronin Y.

115. The method of claim 101, wherein said interface is comprised of a solid substrate, a cell surface or a liposome surface.

116. The method of claim 101, wherein said biological components or reagents are delivered to a solid surface, an array on the surface, or specific elements within said array using microfluid channels or capillary electrophoresis.

117. The method of claim 101, wherein said surface supports a phospholipid bilayer.

118. The method of claim 101, wherein said probe is a virus attached to said solid substrate.

119. The method of claim 101, wherein said binding is an adsorption process of said target onto said solid substrate.

120. The method of claim 101, wherein said binding is a nucleic acid hybridization, wherein said probe and target components are nucleic acids, oligonucleotides, RNA or DNA.

121. The method of claim 101, wherein said probe is a cell surface and said target is a virus binding to said cell surface.

122. A method for optically imaging a surface using a surface-selective nonlinear optical technique, said method comprising illuminating and collecting radiation from said surface, said surface or a component attached to said surface being labeled with a nonlinear optical-active moiety.

123. The method of claim 122, wherein said surface comprises attached probes.

124. The method and apparatus of claim 122 wherein said surface is biological tissue in-situ, in-vivo or in-vitro.

125. The method of 122 wherein said imaging comprises a type of endoscopy.

126. The method of claim 122, wherein said illumination and collection of radiation is achieved using a fiber-optic line.

127. A method for measuring an interaction at an interface between an attached probe and a labelled target, said target being labelled with a biological component at a cell, liposome or supported bilayer surface comprising ion channels, said method comprising measuring changes in the ion properties leading to changes in the nonlinear properties of said labels, said changes in said nonlinear properties of said labels being detected using a surface-selective nonlinear optical technique.

128. The method of claim 127, wherein said changes in the nonlinear properties of said labels comprise a change in hyperpolarizability or wavelength of said labels.

129. The method of claim 127, wherein said changes in the ion channel properties comprise a ligand-receptor binding.

130. The method of claim 127, wherein said changes in the ion channel properties leads to a change in the electric potential or charge density of said cell, liposome, or supported bilayer surface.

131. The method of claim 1, wherein said effects are measured by one or more properties comprising one or more of the following:
i) the intensity of the nonlinear or fundamental light.
ii) the wavelength or spectrum of the nonlinear or fundamental light.
iii) position of incidence of the fundamental light on the surface or substrate.
iv) the time-course of i), ii) or iii).

132. The method of claim 3, wherein said biological cells, liposomes, vesicles, beads, particles are suspended or dissolved in a liquid.
